SEQUENCE LISTING (45 sequences)

SEQ ID NO: 1 - DNA, 440 bp, Blautia sp.
cctacgggtggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtc

SEQ ID NO: 2 - DNA, 460 bp, Bacteroides sp.
cctacggggggctgcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccccagtagtc

SEQ ID NO: 3 - DNA, 439 bp, Unknown
  Description of Unknown Clostridiales sequence
cctacggggggctgcagtggggaatattgcacaatgggcgaaagcctgatgcagcaacgccgcgtgagcgaagaaggtcttcggatcgtaaagctctgtccttggggaagataatgacggtacccttggaggaagccccggctaactacgtgccagcagccgcggtaatacgtagggggcaagcgttatccggaattattgggcgtaaagagtgcgtaggtggttacctaagcagggggtgaaaggcactggcttaaccaatgtcagccccctgaactgggtaccttgagtgcaggagaggaaagcggaattcctagtgtagcggtgaaatgcgtagatattaggaggaacaccagtggcgaaggcggctttctggactgttactgacactgaggcacgaaagtgtggggagcaaacaggattagataccccagtagtc

SEQ ID NO: 4 - DNA, 440 bp, Faecalibacterium sp.
cctacggggggctgcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaagacaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccccagtagtc

SEQ ID NO: 5 - DNA, 442 bp, Oscillibacter sp.
cctacggggggctgcagtggggaatattgggcaatggacgcaagtctgacccagcaacgccgcgtgaaggaagaaggctttcgggttgtaaacttcttttgtcagggaacagtagaagagggtacctgacgaataagccacggctaactacgtgccagcagccgcggtaatacgtaggtggcaagcgttgtccggatttactgggtgtaaagggcgtgcagccgggctggcaagtcaggcgtgaaatcccagggctcaaccctggaactgcgtttgaaactgctggtcttgagtaccggagaggtcatcggaattccttgtgtagcggtgaaatgcgtagatataaggaagaacaccagtggcgaaggcggatgactggacggcaactgacggtgaggcgcgaaagcgtggggagcaaacaggattagataccccggtagtc

SEQ ID NO: 6 - DNA, 440 bp, Unknown
  Description of Unknown Lachnospiracea incertae sedis sequence
cctacggggggctgcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccccagtagtc

SEQ ID NO: 7 - DNA, 440 bp, Unknown
  Description of Unknown Lachnospiraceae sequence
cctacggggggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtaggcggtctgacaagtcagaagtgaaagcccggggctcaactccgggactgcttttgaaactgccggactagattgcaggagaggtaagtggaattcctagtgtagcggtgaaatgcgtagatattaggaggaacaccagtggcgaaggcggcttactggactgtaaatgacgctgaggctcgaaagcgtggggagcaaacaggattagatacccgtgtagtc

SEQ ID NO: 8 - DNA, 440 bp, Unknown
  Description of Unknown Lachnospiraceae sequence
cctacgggtggctgcagtggggaatattgcacaatgggggaaaccctgatgcagcaacgccgcgtgagtgaagaagtatttcggtatgtaaagctctatcagcaggaaagaaaatgacggtacctgactaagaagccccggctaactacgtgccagcagccgcggtaatacgtagggggcaagcgttatccggatttactgggtgtaaagggagcgtagacggtgaggcaagtctgaagtgaaatgccggggctcaaccccggaactgctttggaaactgtcgtactagagtgtcggaggggtaagcggaattcctagtgtagcggtgaaatgcgtagatattaggaggaacaccagtggcgaaggcggcttgctggactgtaactgacactgaggctcgaaagcgtggggagcaaacaggattagatacccttgtagtc

SEQ ID NO: 9 - DNA, 440 bp, Unknown
  Description of Unknown Ruminococcaceae sequence
cctacggggggctgcagtggggaatattgcacaatggaggaaactctgatgcagcgacgccgcgtgagggaagaaggtcttcggattgtaaacctctgttgtcagggacgatgatgacggtacctgacgaggaagccacggctaactacgtgccagcagccgcggtaaaacgtaggtggcaagcgttgtccggaattactgggtgtaaagggagcgcaggcgggagagcaagttgggagtgaaatctgtgggctcaacccacaaattgctttcaaaactgtttttcttgagtggtgtagaggtaggcggaattcccggtgtagcggtggaatgcgtagatatcgggaggaacaccagtggcgaaggcggcctactgggcactaactgacgctgaggctcgaaagcatgggtagcaaacaggattagataccccggtagtc

SEQ ID NO: 10 - DNA, 440 bp, Ruminococcus sp.
cctacggggggctgcagtggggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaacccggggctcaaccccgggactgcattggaaactgttgatctggagtgccggagaggtaagcggaattcctagtgtagcggtgaaatgcgtagatattaggaagaacaccagtggcgaaggcggcttgctggacagtaactgacgttcaggctcgaaagcgtggggagcaaacaggattagatacccttgtagtc

SEQ ID NO: 11 - DNA, 440 bp, Unknown
  Description of Unknown Lachnospiraceae sequence
cctacgggtggcagcagtggggaatattgcacaatgggggaaaccctgatgcagcaacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgcaggcggtacggcaagtcagatgtgaaaacccggggctcaaccccgggactgcatttgaaactgtcggactagagtgccggagaggtaagtggaattcctagtgtagcggtgaaatgcgtagatattaggaggaacaccagtggcgaaggcggcttactaaaccataactgacactgaagcacgaaagcgtggggagcaaacaggattagatacccgggtagtc

SEQ ID NO: 12 - DNA, 445 bp, Bifidobacterium sp.
cctacggggggctgcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccccagtagtc

SEQ ID NO: 13 - DNA, 440 bp, Coprococcus sp.
cctacggggggcagcagtggggaatattgcacaatgggggaaaccctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggtgagacaagtctgaagtgaaaatccggggcttaaccccggaactgctttggaaactgcctgactagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtc

SEQ ID NO: 14 - DNA, 441 bp, Unknown
  Description of Unknown Clostridiales sequence
cctacggggggcagcagtcgggaatattgcgcaatggaggaaactctgacgcagtgacgccgcgtataggaagaaggttttcggattgtaaactattgtcgttagggaagatacaagacagtacctaaggaggaagctccggctaactacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcgactttctggacaataactgacgttgaggcacgaaagtgtggggagcaaacaggattagataccccagtagtc

SEQ ID NO: 15 - DNA, 459 bp, Paraprevotella sp.
cctacggggggcagcagtgaggaatattggtcaatgggcgggagcctgaaccagccaagtagcgtgaaggacgacggccctacgggttgtaaacttcttttataagggaataaagtgcgttacgtgtaatgttttgtatgtaccttatgaataagcatcggctaattccgtgccagcagccgcggtaatacggaagatgcgagcgttatccggatttattgggtttaaagggagcgtaggcgggcttttaagtcagcggtcaaatgtcacggctcaaccgtggccagccgttgaaactgcaagccttgagtctgcacagggcacatggaattcgtggtgtagcggtgaaatgcttagatatcacgaagaactccgatcgcgaaggcattgtgccggggcagcactgacgctgaggctcgaaagtgcgggtatcaaacaggattagatacccctgtagtc

SEQ ID NO: 16 - DNA, 460 bp, Bacteroides sp.
cctacgggaggcagcagtgaggaatattggtcaatgggcgatggcctgaaccagccaagtagcgtgaaggatgactgccctatgggttgtaaacttcttttataaaggaataaagtcgggtatgcatacccgtttgtatgtaccttatgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcctgctaagctgcaactgacattgaggctcgaaagtgtgggtatcaaacaggattagatacccgagtagtc

SEQ ID NO: 17 - DNA, 440 bp, Unknown
  Description of Unknown Ruminococcaceae sequence
cctacggggggctgcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttatccggatttactgggtgtaaagggagcgcaggcgggactgcaagttggatgtgaaataccgtggcttaaccacggaactgcatccaaaactgtagttcttgagtgaagtagaggcaagcggaattccgagtgtagcggtgaaatgcgtagagatggggaggaacaccagtggcgaaggcggcctgctgggctttaactgacgctgaggcacgaaagcgtgggtagcaaacaggattagataccccagtagtc

SEQ ID NO: 18 - DNA, 440 bp, Gemmiger sp.
cctacgggaggcagcagtgggggatattgcacaatgggggaaaccctgatgcagcgacgccgcgtggaggaagaaggttttcggattgtaaactcctgtcgttagggacgataatgacggtacctaacaagaaagcaccggctaactacgtgccagcagccgcggtaaaacgtagggtgcaagcgttgtccggaattactgggtgtaaagggagcgcagacggcactgcaagtctgaagtgaaagcccggggctcaaccccggtactgcattggaaactgtcgtactagagtgtcggaggggtaagcggaattcctagtgtagcggtgaaatgcgtagatatcgggaggaacaccagtggcgaaggcgacctactgggcaccaactgacgctgaggctcgaaagcatgggtagcaaacaggattagatacccctgtagtt

SEQ ID NO: 19 - DNA, 442 bp, Unknown
  Description of Unknown Ruminococcaceae sequence
cctacggggggctgcagtggggaatattaggcaatgggcgaaagcctgacctagcgacgccgcgtgagggaagacggtcttcggattgtaaacctctgtcttcagggacgaagaagatgacggtacctgaagaggaagccacggctaactacgtgccagcagccgcggtaatacgtaggtggcgagcgttgtccggaattactgggtgtaaagggagcgtaggcgggtacgcaagttgaatgtgaaaactaacggctcaaccgatagttgcgttcaaaactgcggatcttgagtgaagtagaggcaggcggaattcctagtgtagcggtaaaatgcgtagatattaggaggaacaccagtggcgaaggcggcctgctgggctttaactgacgctgaggctcgaaagtgtggggagcaaacaggattagataccccggtagtc

SEQ ID NO: 20 - DNA, 466 bp, Dialister sp.
cctacggggggctgcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtgacaagcgttgtccggatttactgggtgtaaagggcgcgtaggcggactgtcaagtcagtcgtgaaataccggggcttaaccccggggctgcgattgaaactgacagccttgagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagatacccgggtagtc

SEQ ID NO: 21 - DNA, 443 bp, Unknown
  Description of Unknown Clostridiales sequence
cctacgggtggctgcagtgggggatattgcacaatggagggaactctgatgcagcaacgccgcgtgaaggacgaaggccttcgggttgtaaacttctgtccttggtgacgaagaaagtgacggtagccagggaggaagccacggctaactacgtgccagcagccgcggtaatacgtaggtggcgagcgttgtccggaattactgggtgtaaagggtgcgtaggcggcttctaaagtcagatgtgaaataccgcagctcaactgcggggctgcatttgaaacttgggagcttgagtgaagtagaggtaagcggaattcctagtgtagcggtggaatgcgtagatattaggaggaacaccagtggcgaaggcggcttactgggctttaactgacgctgaggcacgaaagcgtggggagcaaacaggattagataccccagtagtc

SEQ ID NO: 22 - DNA, 441 bp, Unknown
  Description of Unknown Clostridiales sequence
cctacgggaggctgcagtggggaatattgggcaatgggcgaaagcctgacccagcaacgccgcgtgaaggaagaaggccttcgggttgtaaacttcttttaagagggacgaagaagtgacggtacctcttgaataagccacggctaactacgtgccagcagccgcggtaatacgtaggtggcaagcgttgtccggatttattgggtgtaaagggagcgcagacggcactgcaagtctgaagtgaaagcccggggctcaaccccgggactgctttggaaactgtagagctagagtgctggagaggcaagcggaattcctagtgtagcggtgaaatgcgtagatattaggaggaacaccagtggcgaaggcggcttactggacggtaactgacgttgaggctcgaaagcgtggggagcaaacaggattagatacccgtgtagc

SEQ ID NO: 23 - DNA, 442 bp, Unknown
  Description of Unknown Clostridiales sequence
cctacgggtggctgcagtgggggatattgcgcaatgggggcaaccctgacgcagcaacgccgcgtgaaggaagaaggctttcgggttgtaaacttcttttgtcggggacgaaacaaatgacggtacccgacgaataagccacggctaactacgtgccagcagccgcggtaatacgtagggggctagcgttatccggaattactgggcgtaaagggtgcgtaggtggtttcttaagtcagaggtgaaaggctacggctcaaccgtagtaagcctttgaaactgggaaacttgagtgcaggagaggagagtggaattcctagtgtagcggtgaaatgcgtagatattaggaggaacaccagttgcgaaggcggctctctggactgtaactgacactgaggcacgaaagcgtggggagcaaacaggattagataccctagtagtc

SEQ ID NO: 24 - DNA, 440 bp, Unknown
  Description of Unknown Lachnospiraceae sequence
cctacgggaggcagcagtggggaatattgcacaatgggggaaaccctgatgcagcgacgccgcgtgaaggatgaagtatttcggtatgtaaagctctatcagtagggaagaaaatgacggtacctgactaagaagcaccggctaaatacgtgccagcagccgcggtaatacgtatggtgcaagcgttatccggatttactgggtgtaaaggaagtgtaggtggccaggcaagtcagaagtgaaagcccggggctcaaccccgggactgcttttgaaactgcagggctagagtgcaggagaggtaagtggaattcctagtgtagcggtgaaatgcgtagatattaggaggaacaccagtggcgaaggcggcttgctggacgatgactgacgttgaggctcgaaagcgtggggagcaaacaggattagataccctagtagtc

SEQ ID NO: 25 - DNA, 441 bp, Faecalibacterium sp.
cctacggggggctgcagtgagggatattgggcaatgggggaaaccctgacccagcgacgccgcgtgagggaagacggtcttcggattgtaaacctctgtctttggggacgaaaaaggacggtacccaaggaggaagctccggctaactacgtgccagcagccgcggtaatacgtagggagcgagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatattcggaggaacaccagtggcgaaggcggcctactgggctttaactgacgctgaggctcgaaagtgtggggagcaaacaggattagataccccggtagtc

SEQ ID NO: 26 - DNA, 443 bp, Unknown
  Description of Unknown Clostridiales sequence
cctacgggaggctgcagtggggaatattgcacaatgggggaaaccctgatgcagcaacgccgcgtgaaggatgacggttttcggattgtaaacttcttttcttagtgacgaagacagtgacggtagctaaggaataagcatcggctaactacgtgccagcagccgcggtaatacgtaggatgcaagcgttatccggatttactgggtgtaaagggagcgtaggtggcgaggcaagccagaagtgaaaacccggggctcaaccgcgggattgcttttggaactgtcatgctagagtgcaggaggggtgagcggaattcctagtgtagcggtgaaatgcgtagatattaggaggaacaccagtggcgaaggcggcctactgggcaccaactgacgctgaggctcgaaagtgtgggtagcaaacaggattagataccccggtagtc

SEQ ID NO: 27 - DNA, 465 bp, Megamonas sp.
cctacggggggctgcagtggggaatcttccgcaatgggcgaaagcctgacggagcaacgccgcgtgaacgatgaaggtcttaggatcgtaaagttctgttgttagggacgaagggtaagaatcataataaggtttttatttgacggtacctaacgaggaagccacggctaactacgtgccagcagccgcggtaatacgtaggcggcaagcgttgtccggaattattgggcgtaaagggagcgcaggcgggaaactaagcggatcttaaaagtgcggggctcaaccccgtgatggggtccgaactggttttcttgagtgcaggagaggaaagcggaattcccagtgtagcggtgaaatgcgtagatattgggaagaacaccagtggcgaaggcggctttctggactgtaactgacgctgaggctcgaaagctagggtagcgaacgggattagataccccagtagtc

SEQ ID NO: 28 - DNA, 446 bp, Unknown
  Description of Unknown Coriobacteriaceae sequence
cctacggggggctgcagtggggaatcttgcgcaatggggggaaccctgacgcagcgacgccgcgtgcgggacgaaggccctcgggtcgtaaaccgctttcagcagggaagaggccgaaaggtgacggtacctgcagaagaagccccggctaaatacgtgccagcagccgcggtaatacgtatggggcgagcgttatccggattcattgggcgtaaagcgcgcgtaggcggcctcgtaggccgggagtcaaatccgggggctcaacccccgcccgctcccggaaccccgaggcttgagtctggcaggggagggtggaattcccagtgtagcggtggaatgcgcagatattgggaagaacaccggtggcgaaggcggccctctgggccacgactgacgctgaggcgcgaaagctgggggagcgaacaggattagatacccgagtagtc

SEQ ID NO: 29 - DNA, 440 bp, Unknown
  Description of Unknown Lachnospiraceae sequence
cctacggggggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtaggtggtatggcaagtcagaggtgaaaacccagggcttaaccttgggattgcctttgaaactgtcagactagagtgcaggaggggtaagtggaattcctagtgtagcggtgaaatgcgtagatattaggaggaacaccagtggcgaaggcggcttactggactgtaactgacactgaggctcgaaagcgtggggagcaaacaggattagatacccgagtagtc

SEQ ID NO: 30 - DNA, 442 bp, Unknown
  Description of Unknown Firmicutes sequence
cctacggggggctgcagtggggaatattgggcaatggaggaaactctgacccagcaacgccgcgtggaggaagaaggttttcggatcgtaaactcctgtccttggagacgagtagaagacggtatccaaggaggaagccccggctaactacgtgccagcagccgcggtaatacgtagggggcaagcgttgtccggaataattgggcgtaaagggcgcgtaggcggctcggtaagtctggagtgaaagtcctgcttttaaggtgggaattgctttggatactgtcgggcttgagtgcaggagaggttagtggaattcccagtgtagcggtgaaatgcgtagagattgggaggaacaccagtggcgaaggcgactaactggactgtaactgacgctgaggcgcgaaagtgtggggagcaaacaggattagataccccagtagtc

SEQ ID NO: 31 - DNA, 441 bp, Butyricicoccus sp.
cctacggggggctgcagtggggaatattgcgcaatgggggaaaccctgacgcagcaacgccgcgtgattgaagaaggccttcgggttgtaaagatctttaatcagggacgaaacatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagatacccctgtagtc

SEQ ID NO: 32 - DNA, 440 bp, Unknown; identical to SEQ ID NO: 8
  Description of Unknown Lachnospiraceae sequence

SEQ ID NO: 33 - DNA, 441 bp, Unknown; identical to SEQ ID NO: 14
  Description of Unknown Clostridiales sequence

SEQ ID NO: 34 - DNA, 443 bp, Unknown
  Description of Unknown Ruminococcaceae sequence
cctacggggggctgcagtggggaatattgggcaatgggcgaaagcctgacccagcaacgccgcgtgaaggaagaaggtcttcggattgtaaacttcttttatgagggacgaaggaagtgacggtacctcatgaataagccacggctaactacgtgccagcagccgcggtaatacgtaggtggcaagcgttgtccggatttactgggtgtaaagggcgcgtaggcgggatggcaagtcagatgtgaaatccatgggctcaacccatgaactgcatttgaaactgtcgttcttgagtatcggagaggcaagcggaattcctagtgtagcggtgaaatgcgtagatattaggaggaacaccagtggcgaaggcggcttgctggacgacaactgacgctgaggcgcgaaagcgtggggagcaaacaggattagatacccctgtagtc

SEQ ID NO: 35 - DNA, 440 bp, Unknown
  Description of Unknown Ruminococcaceae sequence
cctacggggggctgcagtgggggatattgcacaatgggggaaaccctgatgcagcaacgccgcgtgagggaagaaggttttcggattgtaaacctctgtcctcagggaagataatgacggtacctgaggaggaagctccggctaactacgtgccagcagccgcggtaatacgtagggagcaagcgttgtccggatttactgggtgtaaagggtgcgtaggcgggatatcaagtcagacgtgaaatccatcggcttaactgatgaactgcgtttgaaactggtattcttgagtgagtcagaggcaggcggaattcccggtgtagcggtgaaatgcgtagagatcgggaggaacaccagtggcgaaggcggcctgctggggcttaactgacgctgaggcacgaaagcgtggggagcaaacaggattagatacccgagtagtc

SEQ ID NO: 36 - DNA, 443 bp, Unknown
  Description of Unknown Firmicutes sequence
cctacggggggctgcagtggggaatattgggcaatggagggaactctgacccagcaatgccgcgtgagtgaagaaggttttcggattgtaaaactctttaagcagggacgaagaaagtgacggtacctgcagaataagcatcggctaactacgtgccagcagccgcggtaatacgtaggatgcaagcgttatccggaatgactgggcgtaaagggtgcgtaggcggtaaatcaagttggcagcgtaattccggggcttaactccggaactactgccaaaactggtgaactagagtgtgtcaggggtaagtggaattcctagtgtagcggtggaatgcgtagatattaggaggaacaccggaggcgaaagcgacttactggggcacaactgacgctgaggcacgaaagcgtggggagcaaacaggattagataccccggtagtc

SEQ ID NO: 37 - DNA, 440 bp, Unknown
  Description of Unknown Ruminococcaceae sequence
cctacgggaggcagcagtgggggatattgcacaatggaggaaactctgatgcagcaacgccgcgtgagggaagaaggatttcggtttgtaaacctctgtcttcggtgacgaaaatgacggtagccgaggaggaagctccggctaactacgtgccagcagccgcggtaatacgtagggagcaagcgttgtccggaattactgggtgtaaagggtgcgtaggtgggactgcaagtcaggtgtgaaaacggtcggctcaaccgatcgcctgcacttgaaactgtggttcttgagtgaagtagaggtaggcggaattcccggtgtagcggtgaaatgcgtagagatcgggaggaacaccagtggcgaaggcggcctactgggctttaactgacgctgaggcacgaaagcatgggtagcaaacaggattagataccccggtagtc

SEQ ID NO: 38 - DNA, 440 bp, Unknown
  Description of Unknown Lachnospiraceae sequence
cctacggggggctgcagtggggaatattgcacaatgggggaaaccctgatgcagcgacgccgcgtgagcgaagaagtatttcggtatgtaaagctctatcagcagggaagataatgacggtacctgactaagaagccccggctaaatacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcggcctactgggcaccaactgacgctgaggctcgaaagtgtgggtagcaaacaggattagataccctagtagtc

SEQ ID NO: 39 - protein, 4 aa, Unknown
  Description of Unknown Metabolite sequence
EEGY

SEQ ID NO: 40 - protein, 4 aa, Unknown
  Description of Unknown Metabolite sequence
RIQI

SEQ ID NO: 41 - protein, 4 aa, Unknown
  Description of Unknown Metabolite sequence
NSHH

SEQ ID NO: 42 - protein, 4 aa, Unknown
  Description of Unknown Metabolite sequence
DFFV

SEQ ID NO: 43 - protein, 4 aa, Unknown
  Description of Unknown Metabolite sequence
HMFF

SEQ ID NO: 44 - DNA, 50 bp, Artificial Sequence
  Description of Artificial Sequence Synthetic primer
  modified_base (42)..(42): a, c, t, or g
tcgtcggcagcgtcagatgtgtataagagacagcctacgggnggcwgcag

SEQ ID NO: 45 - DNA, 55 bp, Artificial Sequence
  Description of Artificial Sequence Synthetic primer
gtctcgtgggctcggagatgtgtataagagacaggactachvgggtatctaatcc

(19) United States
(12) Patent Application Publication
(10) Pub. No.: US 2022/0165361 A1
(43) Pub. Date: May 26, 2022

(54) METHODS OF DIAGNOSING DISEASE

(71) Applicant: 4D Pharma Cork Limited, Cork (IE)

(72) Inventors: PAUL O'TOOLE, Cork (IE); Fergus Shanahan, Cork (IE); Ian Jeffery, Cork (IE); Eileen O'Herlihy, Cork (IE); Anubhav Das, Cork (IE)

(21) Appl. No.: 17/491,564

(22) Filed: Oct. 1, 2021

Related U.S. Application Data
(63) Continuation of application No. PCT/EP2020/059460, filed on Apr. 2, 2020.

(30) Foreign Application Priority Data
Apr. 3, 2019 (EP) 19167114.8
Apr. 3, 2019 (EP) 19167118.9
Jun. 24, 2019 (GB) 1909052.1
Oct. 18, 2019 (GB) 1915143.0
Oct. 18, 2019 (GB) 1915156.2

(51) Int. Cl. G16B 40/20; C12Q 1/689; G16B 30/10; A61K 31/451; A61K 35/741; A61K 35/24; A61P 1/12; G01N 30/72; G16H 50/20

(52) U.S. Cl. CPC G16B 40/20; C12Q 1/689; G16B 30/10; A61K 31/451; A61K 35/741; A61K 35/24; G01N 2030/025; G01N 30/7206; G01N 30/7233; G16H 50/20; G01N 2800/065; G01N 2800/52; A61P 1/12

(57) ABSTRACT

The application provides new and improved methods for diagnosing BAM.

### CROSS-REFERENCE

This application is a continuation of International Application No. PCT/EP2020/059460, filed Apr. 2, 2020, which claims the benefit of European Application No. 19167114.8, filed Apr. 3, 2019, European Application No. 19167118.9, filed Apr. 3, 2019, Great Britain Application No. 1909052.1, filed Jun. 24, 2019, Great Britain Application No. 1915143.0, filed Oct. 18, 2019, and Great Britain Application No. 1915156.2, filed Oct. 18, 2019, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 27, 2021, is named 56686-703_301_SL.txt and is 29,705 bytes in size.

### TECHNICAL FIELD

This invention is in the field of diagnosis and in particular the diagnosis of bile acid malabsorption (BAM).

### BACKGROUND

Bile acid malabsorption (BAM) is a cause of several gut-related problems, in particular chronic diarrhea. It can result from malabsorption secondary to gastrointestinal disease, or be a primary disorder, associated with excessive bile acid production. A proportion of patients incorrectly diagnosed as suffering from diarrhea-predominant irritable bowel syndrome (IBS-D) and alternating or mixed-type irritable bowel syndrome (IBS-M) actually suffer from BAM (1, 2), for which the treatment is different to that for irritable bowel syndrome (IBS) (3). Importantly, BAM is a clinically distinct entity from IBS—not all patients suffering from BAM have IBS, and not all IBS patients suffer from BAM. BAM is estimated to account for 25% to 50% of patients with functional diarrhea or diarrhea-predominant irritable bowel syndrome (IBS-D) and 1% of the population suffer from it (4).

BAM may be treated with bile acid sequestrants. Bile acids are produced in the liver, secreted into the biliary system, stored in the gallbladder and are released after meals stimulated by cholecystokinin. Usually over 95% of the bile acids are absorbed in the terminal ileum and are taken up by the liver and resecreted. When large amounts of bile acids enter the large intestine, they stimulate water secretion and intestinal motility in the colon, which causes symptoms of chronic diarrhea. Bile acid transporters including apical sodium-dependent bile salt transporter (ASBT, IBAT, gene symbol SLC10A2), cytoplasmic ileal bile acid binding protein (IBABP, ILBP, gene symbol FABP6) and the basolateral heterodimer of OSTα and OSTβ transfer bile acids. If expression of these transporters is reduced, the intestine is less able to absorb bile acids (Type 1 bile acid malabsorption). If intestinal motility is affected by gastro-intestinal surgery, or bile acids are deconjugated by small intestinal bacterial overgrowth, absorption is less efficient (Type 3 bile acid malabsorption). Primary bile acid diarrhea (Type 2 bile acid “malabsorption”) may be caused by an overproduction of bile acids. A very small proportion of the patients with no obvious disease (Type 2 bile acid malabsorption) may have mutations in ASBT.

BAM can be diagnosed by the ⁷⁵Selenium (Se) homocholic acid taurine (SeHCAT) test, which detects inability to resorb and metabolize bile acids. The test involves administering a radiolabeled bile compound and measuring its retention after one week (5). BAM is a clinically distinct entity from IBS which can be successfully managed by e.g. with a bile acid sequestrant. The SeHCAT test is the definitive test for BAM diagnosis in the clinic (6) which is expensive and not widely available.

IBS is a common condition that affects the digestive system. Symptoms include cramps, bloating, diarrhoea and constipation and occur over a long time period, generally years. Disorders such as anxiety, major depression, and chronic fatigue syndrome are common among people with IBS. There is no known cure for IBS and treatment is generally carried out to improve symptoms. Treatment may include dietary changes, medication, probiotics, and/or counselling. Dietary measures that are commonly suggested as treatments include increasing soluble fiber intake, a gluten-free diet, or a short-term diet low in fermentable oligosaccharides, disaccharides, monosaccharides, and polyols (FODMAPs). The medication loperamide is used to help with diarrhea while laxatives are be used to help with constipation. Antidepressants may improve overall symptoms and pain. Like most chronic non-communicable disorders, IBS appears to be heterogeneous (7). It ranges in severity from nuisance bowel disturbance to social disablement, accompanied by marked symptomatic heterogeneity (8). Although frequently considered a disorder of the brain-gut axis (9, 10), it is unclear if IBS begins in the gut or in the brain or both. The occurrence of post-infectious IBS (11) suggests that a proportion of cases are initiated in the end-organ, albeit with susceptibility risk factors, some of which may be psychosocial. Advances in microbiome science, with emerging evidence for a modifying influence by the microbiota on neurodevelopment and perhaps on behaviour, have broadened the concept of the mind/body link to encompass the microbiota-gut-brain axis (12). However, progress in understanding and treating IBS has been limited by the absence of reliable biomarkers and IBS is still defined by symptoms. Moreover, the current approach to stratification of patients into clinical subtypes based on predominant symptoms (diarrhea-predominant (IBS-D) or constipation-predominant (IBS-C)) has significant limitations including failure to inform treatment of patients who alternate between subtypes sometimes within days (13). Pharmaceutical agents designed to tackle polar opposite symptoms have the potential for severe unwanted adverse effects if prescribed for a patient misclassified (14).

Investigations have been carried out into gut microbiota alterations in patients with bowel disorders compared to control groups (15-18). Interaction of the microbiome with diet, antibiotics and enteric infections, all of which may be involved in bowel disorders, is consistent with the hypothesis that microbiome alterations could activate or perpetuate pathophysiological mechanisms in the syndrome (19, 20). However, robust microbiome signatures or biomarkers that separate patients with bowel disorders from controls and that help inform therapies are lacking, though signatures have been suggested for IBS severity. Furthermore, most microbiota studies to date have employed 16S rRNA profiling, and did not analyse bacterial metabolites.

There is a requirement for further and improved methods for diagnosing bowel disorders such as BAM. There is also a requirement for further and improved methods for diagnosing bowel disorders such as BAM in patients already diagnosed with another disease with similar symptoms, for example IBS.

### SUMMARY OF THE INVENTION

The inventors have developed new and improved methods for diagnosing bile acid malabsorption (BAM). A comprehensive and detailed analysis of the microbiome and the metabolome in patients and control (non-BAM) individuals has allowed new indicators of disease to be identified. The invention provides a method of diagnosing BAM in a patient comprising detecting: a bacterial species of a taxa associated with BAM and/or a metabolite associated with BAM.

### BRIEF DESCRIPTION OF THE FIGURES

FIGS. 1A-1D. Bile acid malabsorption (BAM) distribution, microbiome and metabolomic profiles in SeHCAT assayed subjects. (FIG. 1A) SeHCAT retention rate in Control and IBS patients. (FIG. 1B) Distribution of BAM classes in IBS-D and IBS-M patients tested. (FIG. 1C) PCoA of the microbiota composition showing no significant difference between BAM classes in IBS patients (Permutational MANOVA with Spearman distance at 16S OTU level; p-value=0.289). (FIG. 1D) PCoA of the fecal MS metabolomics showing a significant difference between BAM classes in IBS patients tested (Permutational MANOVA with Spearman distance at 16S OTU level; p-value<0.001).

FIG. 2. Prediction probabilities for BAM, based on fecal metabolites and Random Forest, on SeHCAT assayed subjects (IBS: n=45; Control n=9).

FIG. 3: Core workflow of an alternative machine learning pipeline. N represents number of features returned by Least Absolute Shrinkage and Selection Operator (LASSO).

FIG. 4: Prediction probabilities for BAM, based on fecal metabolites and Random Forest, on SeHCAT assayed IBS patients (IBS-BAM: n=19; IBS non-BAM n=21). IBS patients with borderline BAM were excluded from the model.

### DISCLOSURE OF THE INVENTION

As shown in the example, the inventors have developed methods for diagnosing bile acid malabsorption (BAM) that are effective and significantly cheaper, more accessible and safer than the ⁷⁵Selenium (Se) homocholic acid taurine (SeHCAT) test. The SeHCAT test is the technique that is currently most widely used for diagnosing BAM, but it exposes patients to radiation, requires a clinical setting and is very expensive, unlike the methods of the invention. In the SeHCAT test, a capsule containing radiolabelled ⁷⁵SeHCAT (with 370 kBq of Selenium-75 and less than 0.1 mg SeHCAT) is administered orally with water. Measurements are taken using an uncollimated gamma camera 1-3 hours after taking the capsule and then at 7 days. The percent retention of SeHCAT at 7 days is then calculated, with a 7-day SeHCAT retention value greater than 15% considered to be normal, and with values less than 15% signifying excessive bile acid loss, as found in bile acid malabsorption.

In one embodiment, the present invention provides a method for diagnosing patients with BAM. In a particular embodiment, the present invention provides a method for diagnosing patients with mild BAM. In a particular embodiment, the present invention provides a method for diagnosing patients with moderate BAM. Moderate BAM may be characterised by retention of 10% of the labelled bile acid analogue in the SeHCAT test. In a particular embodiment, the present invention provides a method for diagnosing patients with severe BAM. The data show that the methods of the invention are particularly effective for diagnosing severe BAM. Severe BAM may be characterised by retention of less than or equal to 5% of the labelled bile acid analogue in the SeHCAT test. In some embodiments, the method of the invention is for diagnosing BAM in patients that have been diagnosed with IBS. In some embodiments, the method of the invention is for diagnosing BAM in patients that have been diagnosed with IBS-M. In some preferred embodiments, the method of the invention is for diagnosing BAM in patients that have been diagnosed with IBS-D. In a particular embodiment, the method of the invention is for diagnosing severe BAM in patients that have been diagnosed with IBS.

In one embodiment, the method comprises diagnosing a patient as suffering from severe BAM based on their microbiota composition. In a particular embodiment, patients suffering from IBS and severe BAM have a distinct microbiota composition. In a particular embodiment, IBS patients suffering from severe BAM have a distinct microbiota composition to IBS patients with normal, mild, moderate or borderline BAM diagnoses.

In one embodiment, the present invention provides a method for diagnosing patients with BAM, comprising detecting a distinct fecal metabolome signature. In a particular embodiment, the present invention provides a method for diagnosing IBS patients with severe BAM, comprising detecting a distinct fecal metabolome signature. In one embodiment, machine learning is applied to fecal metabolome data to predict BAM.

In one embodiment, the present invention provides a method for diagnosing patients with BAM, comprising detecting one or more metabolites predictive of BAM. Generally, detecting a metabolite predictive of BAM or associated with BAM in the methods of the invention comprises measuring the concentration of the metabolite in a sample or measuring changes in the concentration of a metabolite and optionally comparing the concentration to a corresponding sample from a control (non-BAM) individual or relative to a reference value. In a particular embodiment, detecting a metabolite predictive of BAM or associated with BAM in the methods of the invention comprises measuring the concentration of the metabolite in a sample or measuring changes in the concentration of a metabolite and comparing the concentration to a corresponding sample from a patient suffering from IBS. In one embodiment, the one or more metabolites predictive of BAM are selected from: PG(P-16:0/14:0), 2-Ethylsuberic acid, Glu-Glu-Gly-Tyr, 1,2,3-Tris(1-ethoxyethoxy)propane, PG(O-30:1), Ursodeoxycholic acid (UDCA), MG(22:2(13Z,16Z)/0:0/0:0), L-Lysine, O-Phosphoethanolamine, PE(22:5(7Z,10Z,13Z,16Z,19Z)/24:0) and/or Heptadecanoic acid. In another embodiment, the one or more metabolites predictive of BAM are selected from: 1,3-di-(5Z,8Z,11Z,14Z,17Z-eicosapentaenoyl)-2-hydroxy-glycerol (d5), Dimethyl benzyl carbinyl butyrate, 1-18:0-2-18:2-monogalactosyldiacylglycerol, PG(P-16:0/14:0), Glu-Glu-Gly-Tyr, PC(22:2(13Z,16Z)/15:0), PG(34:0), PE(18:3(6Z,9Z,12Z)/P-18:0), MG(22:2(13Z,16Z)/0:0/0:0), Arg-Ile-Gln-Ile, PE(22:5(7Z,10Z,13Z,16Z,19Z)/24:0), PC(18:1(9Z)/15:0), Thiophanate-methyl, Asn-Ser-His-His, 1,2,3-Tris(1-ethoxyethoxy)propane, PS(39:6), 2-Hydroxylauroylcarnitine, Hypoxanthine, Adenosine, PC(40:6), Asp-Phe-Phe-Val, 3-Dehydroxycarnitine, Inosine, PG(O-34:3), 11-Deoxocucurbitacin I, Methyl caprate, Linoleoyl ethanolamide, His-Met-Phe-Phe, 1-Decanol, Gravelliferone, Uridine, Arachidyl carnitine, Guanosine, Methyl nonylate, 3-Epidemissidine, Momordol, N-[2-(1H-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E,17Z)-(1S,3R)-26,27-dimethyl-9,10-seco-5,7,10(19),17(20)-cholestatetraen-22-yne-1,3,25-triol, N-Methylindolo[3,2-b]-5alpha-cholest-2-ene and gamma-Glutamyl-S-methylcysteinyl-beta-alanine. In a preferred embodiment, the method comprises detecting ursodeoxycholic acid. In a preferred embodiment, the method comprises detecting L-lysine. In a preferred embodiment, the method comprises detecting 1,3-di-(5Z,8Z,11Z,14Z,17Z-eicosapentaenoyl)-2-hydroxy-glycerol (d5). In a preferred embodiment, the method comprises detecting Dimethyl benzyl carbinyl butyrate. In a preferred embodiment, the method comprises detecting 1-18:0-2-18:2-monogalactosyldiacylglycerol. In a preferred embodiment, the method comprises detecting PG(P-16:0/14:0). In a preferred embodiment, the method comprises detecting Glu-Glu-Gly-Tyr. In any such embodiments, detecting the metabolite comprises measuring the relative concentration of the metabolite in a sample, for example relative to a corresponding sample from a control (non-BAM) individual or relative to a reference value. In a preferred embodiment, detecting the metabolite comprises measuring the relative concentration of the metabolite in a sample, for example relative to a corresponding sample from a patient suffering from IBS. In some embodiments, the method comprises detecting a precursor or breakdown product of the above metabolites.

In one embodiment, the present invention provides a method for diagnosing patients with BAM, comprising detecting an increase in the concentration of one or more metabolites predictive of BAM. In some embodiments, detecting a metabolite predictive of BAM or associated with BAM in the methods of the invention comprises measuring the concentration of the metabolite in a sample and optionally comparing the concentration to a corresponding sample from a control (non-BAM) individual or relative to a reference value. In some embodiments, metabolites that are predictive of BAM have a higher concentration compared to a corresponding sample from a control (non-BAM) individual or relative to a reference value. In a particular embodiment, detecting a metabolite predictive of BAM or associated with BAM in the methods of the invention comprises measuring the concentration of a metabolite and comparing the concentration to a corresponding sample from a patient suffering from IBS. In some embodiments, metabolites that are predictive of BAM have a higher concentration compared to a corresponding sample from a patient suffering from IBS. In a particular embodiment, the one or more metabolites that are predictive of BAM are selected from: 1,3-di-(5Z,8Z,11Z,14Z,17Z-eicosapentaenoyl)-2-hydroxy-glycerol (d5), Dimethyl benzyl carbinyl butyrate, PG(P-16:0/14:0), PG(34:0), PE(18:3(6Z,9Z,12Z)/P-18:0), Thiophanate-methyl, PS(39:6), Asp-Phe-Phe-Val, PG(O-34:3), 1-Decanol, 3-Epidemissidine and/or Momordol.

In one embodiment, the present invention provides a method for diagnosing patients with BAM, comprising detecting a decrease in the concentration of one or more metabolites predictive of a control individual (non-BAM). In some embodiments, detecting a metabolite predictive of BAM or associated with BAM in the methods of the invention comprises measuring the concentration of the metabolite in a sample and optionally comparing the concentration to a corresponding sample from a control (non-BAM) individual or relative to a reference value. In some embodiments, metabolites that are predictive of BAM have a lower concentration compared to a corresponding sample from a control (non-BAM) individual or relative to a reference value. In a particular embodiment, detecting a metabolite predictive of BAM or associated with BAM in the methods of the invention comprises measuring the concentration of a metabolite and comparing the concentration to a corresponding sample from a patient suffering from IBS. In some embodiments, metabolites that are predictive of BAM have a lower concentration compared to a corresponding sample from a patient suffering from IBS. In a particular embodiment, the one or more metabolites that are predictive of a control (non-BAM) individual are selected from: 1-18:0-2-18:2-monogalactosyldiacylglycerol, Glu-Glu-Gly-Tyr, PC(22:2(13Z,16Z)/15:0), MG(22:2(13Z,16Z)/0:0/0:0), Arg-Ile-Gln-Ile, PE(22:5(7Z,10Z,13Z,16Z,19Z)/24:0), PC(18:1(9Z)/15:0), Asn-Ser-His-His, 1,2,3-Tris(1-ethoxyethoxy)propane, 2-Hydroxylauroylcarnitine, Hypoxanthine, Adenosine, PC(40:6), 3-Dehydroxycarnitine, Inosine, 11-Deoxocucurbitacin I, Methyl caprate, Linoleoyl ethanolamide, His-Met-Phe-Phe, Gravelliferone, Uridine, Arachidyl carnitine, Guanosine, Methyl nonylate, N-[2-(1H-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E,17Z)-(1S,3R)-26,27-dimethyl-9,10-seco-5,7,10(19),17(20)-cholestatetraen-22-yne-1,3,25-triol, N-Methylindolo[3,2-b]-5alpha-cholest-2-ene and/or gamma-Glutamyl-S-methylcysteinyl-beta-alanine.

In some embodiments, detecting a metabolite associated with BAM in the methods of the invention comprises measuring the concentration of a precursor of the metabolite and optionally comparing the concentration to a corresponding sample from a control (non-BAM) individual or relative to a reference value. In some embodiments, detecting a metabolite associated with BAM in the methods of the invention comprises measuring the concentration of a breakdown product of the metabolite and optionally comparing the concentration to a corresponding sample from a control (non-BAM) individual or relative to a reference value. In certain embodiments, the method comprises detecting a bacterial taxa known to produce a metabolite predictive of BAM.

In one embodiment, the present invention provides a method for diagnosing patients with BAM, comprising detecting metabolites which are predictive of BAM selected from table 1 and/or table 7. In a particular embodiment, the present invention provides a method for diagnosing IBS patients with severe BAM, comprising detecting metabolites which are predictive of BAM selected from table 1 and/or table 7. In preferred embodiments, the method of the invention comprises detecting metabolites associated with fatty acid metabolism. In preferred embodiments, the method of the invention comprises detecting ursodeoxycholic acid. In one embodiment, machine learning is used to diagnose BAM. In any such embodiments, detecting the metabolite comprises measuring the relative concentration of the metabolite in a sample, for example relative to a corresponding sample from a control (non-BAM) individual or relative to a reference value.

The inventors have identified bacterial taxa that are associated with BAM, as demonstrated in the example. Accordingly, the invention provides methods for diagnosing BAM comprising detecting the presence of certain bacterial taxa. Preferably, these methods comprise detecting bacterial strains in a fecal sample from a patient. Alternatively, the bacteria (i.e. one or more bacterial strains) may be detected from an oral sample, such as a swab. Generally, detecting a bacterial taxa associated with BAM in the methods of the invention comprises measuring the relative abundance of the bacteria (i.e. one or more bacterial strains) in a sample, for example relative to a corresponding sample from a control (non-BAM) individual or relative to a reference value.

In one embodiment, the invention provides a method for diagnosing BAM, comprising detecting bacterial species of one or more of the following families: Lachnospiraceae, Bacteroidaceae, Ruminococcaceae, Bifidobacteriaceae, Prevotellaceae, Veillonellaceae and Coriobacteriaceae. In one embodiment, the present invention provides a method for diagnosing BAM, comprising detecting bacterial species of one or more of the following genera: Blautia, Bacteroides, Faecalibacterium, Oscillibacter, Ruminococcus, Bifidobacterium, Coprococcus, Paraprevotella, Gemmiger, Dialister and Megamonas. In any such embodiments, detecting the bacteria (i.e. one or more bacterial strains) comprises measuring the relative abundance of the bacteria in a sample, for example relative to a corresponding sample from a control (non-BAM) individual or relative to a reference value. The examples demonstrate that methods detecting these bacteria are particularly effective. The bacterial taxa used in the invention may be defined with reference to 16S rRNA gene sequences, or the invention may use Linnaean taxonomy. Bacteria of either category of taxa may be detected using clade-specific bacterial genes, 16S sequences, transcriptomics, metabolomics, or a combination of such techniques. In certain embodiments, the bacteria (i.e. one or more bacterial strains) may be detected using clade-specific bacterial genes, 16S sequences, transcriptomics or metabolomics.

In one embodiment, the present invention provides a method for diagnosing BAM, comprising detecting one or more bacterial strains belonging to an operational taxonomic unit (OTU) associated with BAM. As is well known in the art, an operational taxonomic unit (OTU) is an operational definition used to classify groups of closely related individuals. As used herein, an “OTU” is a group of organisms which are grouped by DNA sequence similarity of a specific taxonomic marker gene (39). In some embodiments, the specific taxanomic marker gene is the 16S rRNA gene. In some embodiments, the Ribosomal Database Project (RDP) taxonomic classifier is used to assign taxonomy to representative OTU sequences. For example, the sequence information in Table 3 can be used to classify whether bacteria (i.e. one or more bacterial strains) belong to the OTUs listed in Table 2.

Bacteria having at least 97% sequence identity to the sequences in Table 3 belong to the corresponding OTUs in Table 2. In preferred embodiments, the OTU is selected from table 2. In any such embodiments, detecting the bacteria (i.e. one or more bacterial strains) comprises measuring the relative abundance of the bacteria in a sample, for example relative to a corresponding sample from a control (non-BAM) individual or relative to a reference value.

In one embodiment, the present invention provides a method for diagnosing BAM, comprising detecting one or more bacterial strains belonging to an operational taxonomic unit (OTU) associated with BAM. In preferred embodiments, the OTU is selected from table 2. In one embodiment, the OTU associated with BAM is classified as belonging to one of the following phyla: Firmicutes, Bacteroidetes or Actinobacteria. In a particular embodiment, the OTU associated with BAM is classified as belonging to one of the following classes: Clostridia, Bacteroidia, Actinobacteria or Negativicutes. In a particular embodiment, the OTU associated with BAM is classified as belonging to one of the following orders: Clostridiales, Bacteroidales, Selenomonadales or Coriobacteriales. In a particular embodiment, the OTU associated with BAM is classified as belonging to one of the following families: Lachnospiraceae, Bacteroidaceae, Ruminococcaceae, Bifidobacteriaceae, Prevotellaceae, Veillonellaceae or Coriobacteriaceae. In a particular embodiment, the OTU associated with BAM is classified as belonging to one of the following genera: Blautia, Bacteroides, Faecalibacterium, Oscillibacter, Lachnospiracea_incertae_sedis, Ruminococcus2, Bifidobacterium, Coprococcus, Paraprevotella, Gemmiger, Dialister or Megamonas.

In one embodiment, the present invention provides a method for diagnosing BAM, comprising detecting bacterial strains belonging to one or more OTUs listed in Table 2. The sequences in Table 3 can be used to classify bacteria (i.e. one or more bacterial strains) as belonging to the OTUs listed in Table 2. Bacteria (i.e. one or more bacterial strains) having at least 97% sequence identity to the sequences in Table 3 belong to the corresponding OTUs in Table 2. The alignment is across the length of the sequence. In both Metaphlan2 and HUMAnN2 runs, alignment for species composition is done using bowtie 2. Bowtie2 is run with “very-sensitive argument” and the alignment performed is “Global alignment”.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 1. In certain such embodiments, the bacteria (i.e. one or more bacterial strains) is classified as belonging to the Blautia genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 2. In certain such embodiments, the bacteria (i.e. one or more bacterial strains) is classified as belonging to the Bacteroides genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 3. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 4. In certain such embodiments, the bacteria (i.e. one or more bacterial strains) is classified as belonging to the Faecalibacterium genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 5. In certain such embodiments, the bacteria (i.e. one or more bacterial strains) is classified as belonging to the Oscillibacter genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 6. In certain such embodiments, the bacteria (i.e. one or more bacterial strains) is classified as belonging to the Lachnospiracea genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 6. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 7. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 8. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 9. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 10. In certain such embodiments, the bacteria (i.e. one or more bacterial strains) is classified as belonging to the Ruminococcus genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 11. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 12. In certain such embodiments, the bacteria (i.e. one or more bacterial strains) is classified as belonging to the Bifidobacterium genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 13. In certain such embodiments, the bacteria (i.e. one or more bacterial strains) is classified as belonging to the Coprococcus genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 14. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 15. In certain such embodiments, the bacteria (i.e. one or more bacterial strains) is classified as belonging to the Paraprevotella genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 16. In certain such embodiments, the bacteria (i.e. one or more bacterial strains) is classified as belonging to the Bacteroides genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 17. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 18. In certain such embodiments, the bacteria (i.e. one or more bacterial strains) is classified as belonging to the Gemmiger genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 19. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 20. In certain such embodiments, the bacteria (i.e. one or more bacterial strains) is classified as belonging to the Dialister genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 21. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 22. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 23. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 24. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 25. In certain such embodiments, the bacteria (i.e. one or more bacterial strains) is classified as belonging to the Faecalibacterium genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 26. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 27. In certain such embodiments, the bacteria (i.e. one or more bacterial strains) is classified as belonging to the Megamonas genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 28. In certain such embodiments, the bacteria (i.e. one or more bacterial strains) is classified as belonging to the Coriobacteriaceae family.

In preferred embodiments, the invention provides a method for diagnosing BAM, comprising detecting different bacteria (i.e. one or more bacterial strains) having 16S rRNA gene sequences at least 97% (e.g. 98%, 99%, 99.5% or 100%) identical to two or more of SEQ ID No:1-28, such as 5, 8, or all of SEQ ID No:1-28.

In one embodiment, the present invention provides a further step of diagnosing IBS, comprising detecting bacterial strains belonging to one or more OTUs listed in Table 5. The sequences in Table 6 can be used to classify bacteria (i.e. one or more bacterial strains) as belonging to the OTUs listed in Table 5. Bacteria (i.e. one or more bacterial strains) having at least 97% sequence identity to the sequences in Table 6 belong to the corresponding OTUs in Table 5. The alignment is across the length of the sequence. In both Metaphlan2 and HUMAnN2 runs, alignment for species composition is done using bowtie 2. Bowtie2 is run with “very-sensitive argument” and the alignment performed is “Global alignment”.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 29. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 30. In certain such embodiments, the bacteria (i.e. one or more bacterial strains) is classified as belonging to the Firmicutes phylum.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 31. In certain such embodiments, the bacteria (i.e. one or more bacterial strains) is classified as belonging to the Butyricicoccus genus.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 32. In certain such embodiments, the bacteria (i.e. one or more bacterial strains) is classified as belonging to the Lachnospiraceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 33. In certain such embodiments, the bacteria (i.e. one or more bacterial strains) is classified as belonging to the Clostridiales order.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 34. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 35. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 36. In certain such embodiments, the bacteria (i.e. one or more bacterial strains) is classified as belonging to the Firmicutes phylum.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No: 37. In certain such embodiments, the bacteria (i.e. one or more bacterial strains) is classified as belonging to the Ruminococcaceae family.

In one embodiment, the present invention provides a method for diagnosing BAM, comprising detecting bacteria (i.e. one or more bacterial strains) having a 16S rRNA gene sequence at least 97% (e.g. 98%, 99%, 99.5% or 100%) identical to SEQ ID No:38. In certain such embodiments, the bacteria (i.e. one or more bacterial strains) is classified as belonging to the Lachnospiraceae family.

In preferred embodiments, the invention provides a method for diagnosing BAM, comprising detecting different bacteria (i.e. one or more bacterial strains) having 16S rRNA gene sequences at least 97% (e.g. 98%, 99%, 99.5% or 100%) identical to two or more of SEQ ID No: 29-38, such as 5, 8, or all of SEQ ID No: 29-38.

In certain embodiments, the bacterial species belongs to a sequence-based taxon. In preferred embodiments, the sequence-based taxon is selected from table 2.

In preferred embodiments, the method for diagnosing BAM comprises detecting at least one metabolite as set out above and detecting at least one bacterial strain or species as set out above. While the metabolomics model performed with 100% accuracy for severe and moderate BAM, the OTU model resulted in fewer misclassifications (five) compared to the fecal metabolomics model (nine). There was no overlap in misclassified subjects between the models, indicating that a combined microbiome-metabolome model would increase BAM prediction accuracy.

In one embodiment, the present invention provides a method for diagnosing BAM in patients already diagnosed with a disease that is co-morbid with BAM. In another embodiment, the diagnosis of BAM using the BAM metabolomic signature distinguishes patients suffering from BAM to patients suffering from other diseases, for example diseases that are co-morbid with BAM.

In a particular embodiment, the present invention provides a method for diagnosing BAM in patients already diagnosed with inflammatory bowel disease, e.g. ulcerative colitis or Crohn's disease. In a particular embodiment, the diagnosis of BAM using the BAM metabolomic signature distinguishes patients suffering from BAM to patients suffering from other diseases, for example inflammatory bowel disease, e.g. ulcerative colitis or Crohn's disease.

In one embodiment, the present invention provides a method for diagnosing BAM in patients already diagnosed with anorexia nervosa. In another embodiment, the diagnosis of BAM using the BAM metabolomic signature distinguishes patients suffering from BAM to patients suffering from other diseases, for example anorexia nervosa.

In certain embodiments, the method for diagnosing BAM comprises detecting one or more bacterial species and one or more metabolite.

Integrative Analysis of Diet, Microbiome and Metabolome in BAM Patients

In certain embodiments, the invention provides a method of diagnosing BAM comprising one or more of i) detecting a bacterial species, for example as discussed above, ii) detecting metabolites, for example as discussed above. In any such embodiments, detecting the bacteria, gene or metabolite comprises measuring the abundance or concentration of said marker in a sample, for example the relative to a corresponding sample from a control (non-BAM) individual or relative to a reference value.

Diagnostic Methods

The inventors have developed new and improved methods for diagnosing BAM.

In preferred embodiments, the methods of the invention are for use in diagnosing a patient resident in Europe, such as Northern Europe, preferably Ireland or a patient that has a European, Northern European or Irish diet. The examples demonstrate that the methods of the invention are particular effective for such patients. In other embodiments, the patient may be resident in the United States of America.

In certain embodiments of any aspect of the invention, the abundance of bacteria, genes or metabolites is assessed relative to control (non-BAM) individuals. Such reference values may be generated using any technique established in the art.

In certain embodiments of any aspect of the invention, comparison to a corresponding sample from a control (non-BAM) individual is a comparison to a corresponding sample from a healthy individual.

Preferably the method of diagnosing BAM has a sensitivity of greater than 40% (e.g. greater than 45%, 50% or 52%, e.g. 53% or 58%) and a specificity of greater than 90% (e.g. greater than 93% or 95%, e.g. 96%).

In certain embodiments, the method of diagnosis is a method of monitoring the course of treatment for BAM.

In certain embodiments, the step of detecting the presence or abundance of bacteria, such as in a fecal sample, comprises a nucleic acid based quantification methodology, for example 16s rRNA gene amplicon sequencing. Methods for qualitative and quantitative determination of bacteria in a sample using 16s rRNA gene amplicon sequencing are described in the literature and will be known to a person skilled in the art. Other techniques may involve PCR, rtPCR, qPCR, high throughput sequencing, metatranscriptomic sequencing, or 16S rRNA analysis.

In alternative aspects of any embodiment of the invention, the invention provides a method for diagnosing the risk of developing BAM.

In any embodiment of the invention, modulated abundance of a bacterial strain, species or metabolite is indicative of BAM. In preferred embodiments, the abundance of the bacterial strain, species or OTU as a proportion of the total microbiota in the sample is measured to determine the relative abundance of the strain, species or OTU. In preferred embodiments, the concentration of a metabolites is measured. In preferred embodiments, the abundance of bacterial strains as a proportion of the total microbiota in the sample is measured to determine the relative abundance of the strains. Then, in such preferred embodiments, the relative abundance of the bacterium or OTU or the concentration of the metabolite or gene sequence in the sample is compared with the relative abundance or concentration in the same sample from a reference control (non-BAM) individual. A difference in relative abundance of the bacterium or OTU in the sample, e.g. a decrease or an increase, compared to the reference is a modulated relative abundance. As explained herein, detection of modulated abundance can also be performed in an absolute manner by comparing sample abundance values with absolute reference values. Therefore, the invention provides a method of determining BAM status in an individual comprising the step of assaying a biological sample from the individual for a relative abundance of one or more BAM-associated bacteria and/or a modulated concentration of a metabolite, wherein a modulated relative abundance of the bacteria or modulated concentration of a metabolite is indicative of BAM. Similarly, the invention provides a method of determining whether an individual has an increased risk of having BAM comprising the step of assaying a biological sample from the individual for a relative abundance of one or more BAM-associated oral bacteria or BAM-associated metabolites, wherein modulated relative abundance or concentration is indicative of an increased risk.

In any embodiment of the invention, detecting bacteria may comprise detecting “modulated relative abundance”. As used herein, the term “modulated relative abundance” as applied to a bacterium or OTU in a sample from an individual should be understood to mean a difference in relative abundance of the bacterium or OTU in the sample compared with the relative abundance in the same sample from a reference control (non-BAM) individual (hereafter “reference relative abundance”). In one embodiment, the bacterium or OTU exhibits increased relative abundance compared to the reference relative abundance. In one embodiment, the bacterium or OTU exhibits decreased relative abundance compared to the reference relative abundance. Detection of modulated abundance can also be performed in an absolute manner by comparing sample abundance values with absolute reference values. In one embodiment, the reference abundance values are obtained from age and/or sex matched individuals. In one embodiment, the reference abundance values are obtained from individuals from the same population as the sample (i.e. Celtic origin, North African origin, Middle Eastern origin). Method of isolating bacteria from oral and fecal sample are described below, as are methods for detecting abundance of bacteria (i.e. one or more bacterial strains). Any suitable method may be employed for isolating specific species or genera of bacteria, which methods will be apparent to a person skilled in the art. Any suitable method of detecting bacterial abundance may be employed, including agar plate quantification assays, fluorimetric sample quantification, qPCR, 16S rRNA gene amplicon sequencing, and dye-based metabolite depletion or metabolite production assays.

The invention also provides kits comprising reagents for performing the methods of the invention, such as kits containing reagents for detecting one or more, such as two or more of the bacterial species, genes or metabolites set out above. Also provided are kits that find use in practicing the subject methods of diagnosing BAM, as mentioned above. The kit may be configured to take a biological sample from an individual, for example a urine sample or a fecal sample. The individual may be suspected of having BAM. The individual may be suspected of being at increased risk of having BAM. A kit can comprise a sealable container configured to receive the biological sample. A kit can comprise polynucleotide primers. The polynucleotide primers may be configured for amplifying a 16S rRNA polynucleotide sequence from at least one BAM-associated bacterium to form an amplified 16S rRNA polynucleotide sequence. A kit may comprise a detecting reagent for detecting the amplified 16S rRNA sequence. A kit may comprise instructions for use.

### EXAMPLES

Summary

Background & Aims: Diagnosis of BAM is based on SeHCAT analysis. Some patients have an alteration in their microbiota. Therefore, microbiome and metabolomic profiling was conducted to identify biomarkers for the condition.

Methods:

Anthropometric, medical and dietary information were collected with fecal samples for microbiome and metabolomic analyses. Shotgun and 16S rRNA amplicon sequencing were performed on feces, and fecal metabolites were analysed by gas chromatography (GC)—and liquid chromatography (LC) mass spectrometry (MS). Bile acid malabsorption (BAM) was identified in patients with diarrhea by retention of radiolabelled ⁷⁵Selenium (Se) homocholic acid taurine (SeHCAT).

Results: BAM was accurately distinguished within IBS by fecal metabolomics.

Conclusion: BAM can be identified by species-, metagenomics and fecal metabolomic-signatures which are from those of IBS. These findings are useful for diagnosing BAM and for developing precision therapeutics for IBS and BAM.

### Example 1—Fecal Microbiome and Metabolome Analysis of IBS Patients with Bile Acid Malabsorption (BAM)

Materials and Methods

Subject recruitment: Eighty patients aged 16-70 years with IBS meeting the Rome IV criteria were recruited at Cork University Hospital. Clinical subtyping of the patients (21) was as follows: IBS with constipation (IBS-C), mixed IBS (IBS-M) or IBS with diarrhea (IBS-D). Sixty-five controls of the same age range and of the same ethnicity and geographic region were recruited. Descriptive statistics for the study population are presented in Table 4.

Exclusion criteria included the use of antibiotics within 6 weeks prior to study enrolment, other chronic illnesses including gastrointestinal diseases, severe psychiatric disease, abdominal surgery other than hernia repair or appendectomy. Standard-of-care blood analysis was carried out on all participants if recent results were not available, and all subjects were tested by serology to exclude coeliac disease. The inclusion/exclusion criteria for the control population were the same as for the IBS population with the exception of having to fulfil the Rome IV criteria for IBS. Gastrointestinal (GI) symptom history, psychological symptoms, diet, medical history and medication data were collected on each participant (both IBS and controls) and using the following questionnaires: Bristol Stool Score (BSS), Hospital Anxiety and Depression Scale (HADS) (22); Food Frequency Questionnaire (FFQ) (23). IBS-D and IBS-M patients reporting diarrhoea as well as a subset of consenting control subjects were assessed for bile acid malabsorption by SeHCAT, a radiolabelled synthetic bile acid which is used to clinically diagnosis of BAM which is not metabolized by bacteria and passes through the enterohepatic circulation as endogenous bile acids. Ethical approval for the study was granted by the Cork Research Ethics Committee (protocol number: 4DC001) before commencing the study and all participants provided written informed consent to take part.

Sample collection: Fecal and urine samples were collected from all participants for microbiome and metabolomics profiling. Subjects collected a freshly voided fecal sample at home using a collection kit and brought the sample to the clinic that day, when a fresh urine sample was collected. Samples were kept at 4° C. until brought to the laboratory for storage at −80° C. which was within a few hours of the sample collection.

Microbiome profiling and metagenomics: Genomic DNA was extracted and amplified from frozen fecal samples (0.25 g) using the method described by Browne et al. (24). The modifications from the methods described by Brown et al (18) included bead beating tubes consisted of 0.5 g of 0.1 mm zirconia beads and 4×3.5 mm glass beads. Fecal samples were homogenised via bead beating for 3×60 s cycles and cooled on ice between each cycle. Genomic DNA was visualised on 0.8% agarose gel and quantified using the SimpliNano Spectrometer (Biochrom™, US). The PCR master mix used 2× Phusion Taq High-Fidelity Mix (Thermo Scientific, Ireland) and 15 ng of DNA. The resulting PCR products were purified, quantified and equimolar amounts of each amplicon were then pooled before being sent for sequencing to the commercial supplier (GATC Biotech AG, Konstanz, Germany) on the MiSeq (2×250 bp) chemistry platforms. Sequencing was performed by GATC Biotech, Germany on an Illumina MiSeq instrument using a 2×250 bp paired end sequencing run.

Microbiome profiling and metagenomics—16S amplicon sequencing: Using the Qiagen DNeasy Blood & Tissue Kit and following the manufacturer's instructions, microbial DNA was extracted from 0.25 g of each of 144 frozen fecal samples (IBS: n=80 and control (n=64). No fecal sample was available for one control subject. The 16S rRNA gene amplicons preparation and sequencing was carried out using the 16S Sequencing Library Preparation Nextera protocol developed by Illumina (San Diego, Calif., USA). 15 ng of each of the DNA fecal extracts was amplified using PCR and primers targeting the V3-V4 variable region of the 16S rRNA gene using the following gene-specific primers:

16S Amplicon PCR Forward Primer
(S-D-Bact-0341-b-S-17) = 5′:
(SEQ ID NO: 44)
TCGTCGGCAGCGTCAGATGTGTATAAGAGACAGCCTACGGGNGGCWGC
AG
16S Amplicon PCR Reverse Primer
(S-D-Bact-0785-a-A-21) = 5′:
(SEQ ID NO: 45)
GTCTCGTGGGCTCGGAGATGTGTATAAGAGACAGGACTACHVGGGTAT
CTAATCC

The amplicon size was 531 bp. The products were purified and forward and reverse barcodes were attached by a second round of adapter PCR.

Microbiome profiling and metagenomics—Shotgun sequencing: Genomic DNA was extracted as described above. The DNA quality was checked on 0.8% agarose gel and quantified using the Simplinano (Thermo Scientific, Ireland). For shotgun sequencing, 1 μg (concentration>5 ng/μl) of high molecular weight DNA for each sample was sent to GATC Biotech, Germany for sequencing on Illumina HiSeq platform (HiSeq 2500) using 2×250 bp paired-end chemistry. This returned 2,714,158,144 raw reads (2,612,201,598 processed reads) of which 45.6% were mapped to an average of 222,945 gene families per sample with a mean count value of 8,924,302±2,569,353 per sample.

Bioinformatics analysis (16S amplicon sequencing): Miseq 16S sequencing data was returned for 144 subjects. Data generated for 3 samples (2 IBS and 1 control) were removed as the number of reads returned from sequencing was too low for analysis, leaving 141 samples (control: n=63, IBS n=78). Raw amplicon sequence data were merged and the reads trimmed using the flash methodology (25). The USEARCH pipeline was used to generate the OTU table (26). The UPARSE algorithm was used to cluster the sequences into OTUs at 97% similarity (27). UCHIME chimera removal algorithm was used with Chimeraslayer to remove chimeric sequences (28). The Ribosomal Database Project (RDP) taxonomic classifier was used to assign taxonomy to the representative OTU sequences (26) and microbiota compositional (abundance and diversity) information was generated.

Bioinformatics analysis (Shotgun metagenomic sequencing): For shotgun metagenomics, 6 control samples were not sequenced due to data not passing QC or no sample available (control: n=59; IBS n=80). The number of raw read pairs obtained after sequencing, varied from 5,247,013 to 21,280,723 (Mean=9,763,159±2,408,048). Reads were processed in accordance with the Standard Operating Procedure of Human Microbiome Project (HMP) Consortium (29). Metagenomic composition and functional profiles were generated using HUMAnN2 pipeline (30). For each sample, multiple profiles were obtained, including: microbial composition profiles from clade-specific gene information (using MetaPhlAn2) and Gene family abundance.

Fecal GC/LC MS: 1 g samples of frozen feces were sent on dry ice to Metabolomic Discoveries (now Metabolon), Potsdam, Germany. For LC-MS, the samples were dried and resuspended to a final concentration of 10 mg per 400 μl before analysis. GC-MS and SCFA analysis were performed using wet samples. Untargeted metabolomics analysis was performed using liquid chromatography (LC) and Solid Phase Microextraction (SPME) gas chromatography (GC) and metabolites were identified using electrospray ionization mass spectrometry (ESI-MS). SCFA analysis was also performed by LC-tandem mass spectrometry.

Bioinformatics analysis of fecal metabolome data: Fecal MS metabolomics data was returned for all IBS subjects (n=80) and all but 2 controls (n=63) as these did not pass QC or no sample was available. 2,933 metabolites were returned from untargeted fecal metabolomics analysis carried out by the service provider of which 753 were identified. Metabolites identified using LC-MS were not normalized, since the fecal samples were already normalized with dry weight (10 mg per 400 μl) during sample preparation. Metabolites identified using GC-MS were normalized with corresponding sample wet weights. Only the identified metabolites were considered for further analyses. Machine learning analysis was carried out as described below. Summary statistics for all datasets were generated using the Wilcoxon rank sum test with q-value adjustment for multiple testing.

BAM SeHCAT assay: SeHCAT was administered at Cork University Hospital as a single capsule dose containing less than 0.1 mg of tauroselcholic acid (GE Healthcare, UK) and with a radioactivity dose of 370 kBq at the reference date. A baseline whole-body absorption reading using an uncollimated gamma counter (Siemens Ecam camera) was taken for each subject 2-3 hours after capsule administration. A follow-up scan was taken 7 days later and the proportion of bile acid retention was calculated; a value of <15% retention indicated mild to severe BAM with a SeHCAT score of 15-20% representing a borderline classification as discussed by Watson et al. (31).

Machine learning: An in-house machine learning pipeline was applied to each datatype (16S, shotgun and BAM-fecal MS metabolomics) using a twostep approach applying the Least Absolute Shrinkage and Selection Operator (LASSO) feature selection followed by Random Forest (RF) modelling (32). The models were implemented using R software version 3.4.0, using package glmnet version 2.0-10 for LASSO feature selection, and RF package randomForest version 4.6-12.

First, feature selection was performed using the LASSO algorithm to improve accuracy and interpretability of models by efficiently selecting the relevant features. This process was tuned by parameter lambda, which was optimized for each dataset using a grid search. The training data was filtered to include only the features selected by the LASSO algorithm, and RF was then used for modelling whereby 1500 trees were built. Both LASSO feature selection and RF modelling were performed using 10-fold cross validation (CV), which generated an internal 10-fold prediction yielding an optimal model that predicts the IBS or Control classification of samples.

For BAM-fecal metabolomics data analysis, machine learning was performed in a similar manner with the only difference being that instead of ten-fold cross-validation, Leave-One-Out (LOO) CV was used at every cross-validation step

Model 1; BAM (borderline to severe BAM or SeHCAT retention <20%) or Normal bile acid (SeHCAT retention >20%) for IBS and control subjects.

Model 2; BAM (mild to severe BAM or SeHCAT retention <15%) or Normal bile acid (SeHCAT retention >20%) for IBS subjects only.

Co-inertia analysis of the data types: The microbiome derived datasets were Hellinger transformed. Co-inertia analysis was performed using ade4 (v. 1.7.2) package in R (v 3.2.0). Principal component analysis (PCA) was performed on each of the profile in the comparison pair, followed by co-inertia analysis on these PCA objects on the first 5 principal axes. Significance of the co-inertia was calculated by permutation test using the randtest function.

Results

IBS Patients with Bile Acid Malabsorption have Altered Fecal Microbiome

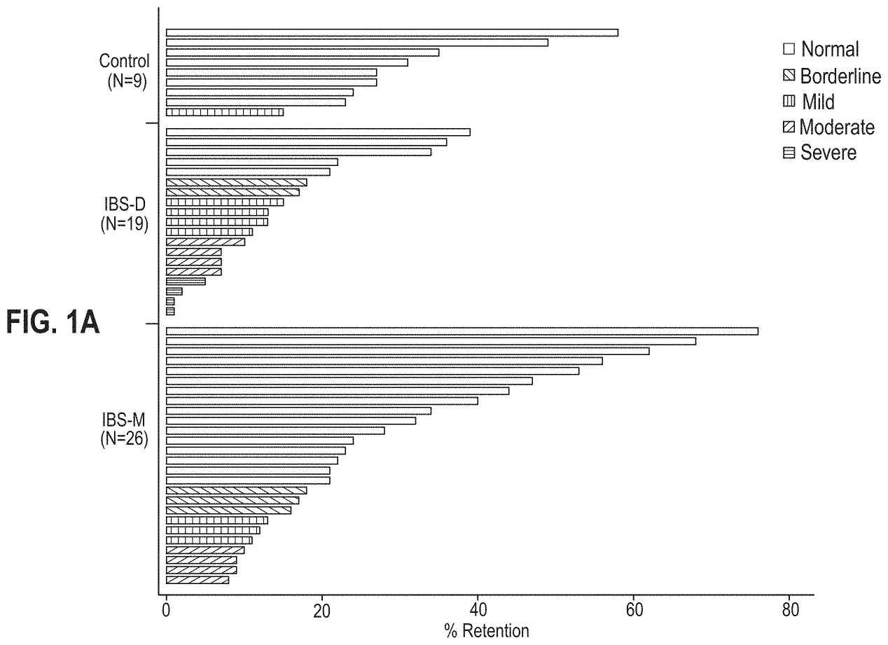

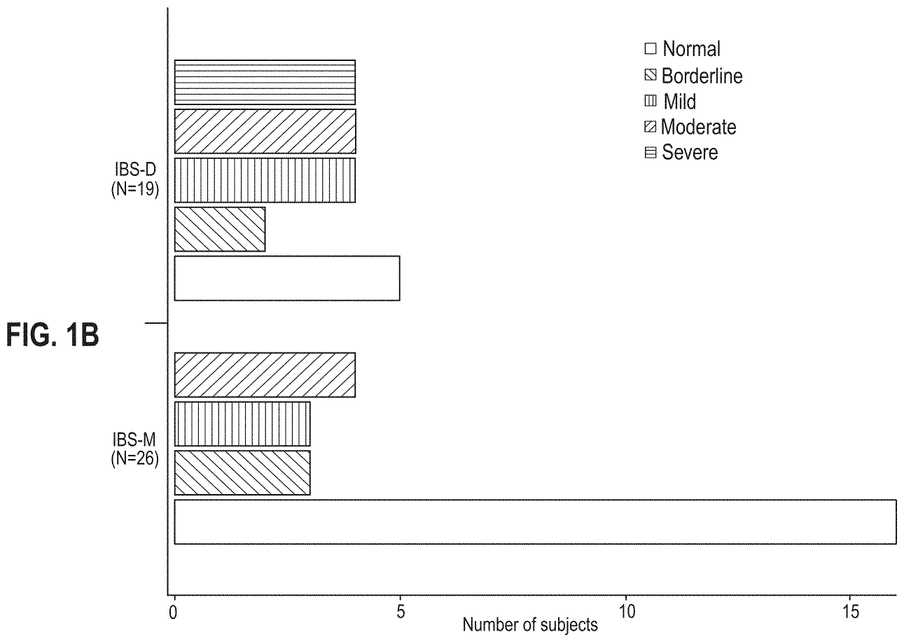

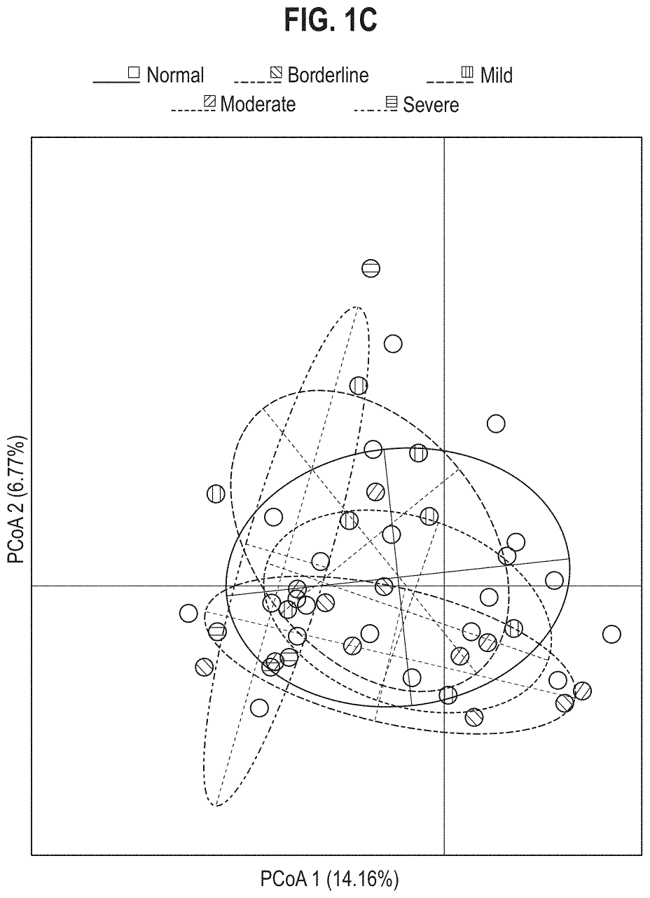

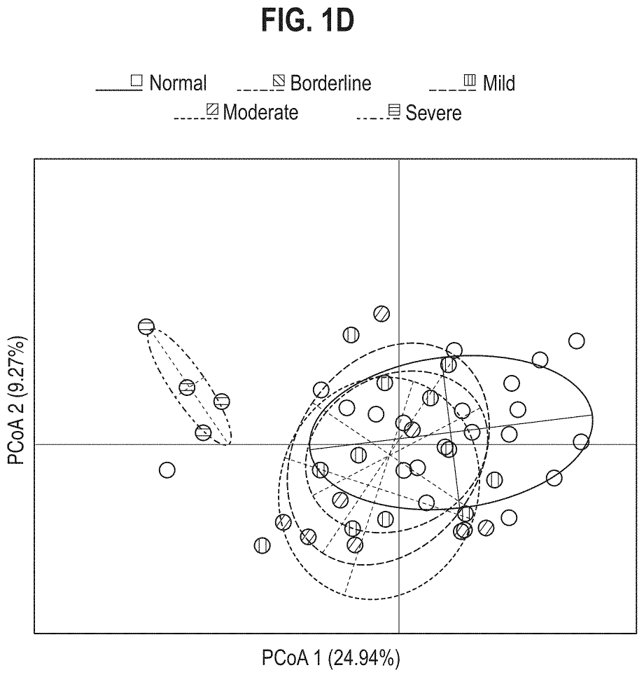

Since some patients with IBS-D may have bile acid malabsorption (BAM) which will influence transit time and possibly microbiota composition, 19/21 patients with IBS-D, 26/29 subjects with IBS-M and 9/65 controls were tested for SeHCAT retention, the gold standard for identifying BAM (33). Failure to retain >15% of the labelled bile acid analogue was classified as BAM, 10-15% retention classified as mild BAM, 5-10% as moderate BAM and <5% as severe BAM (FIG. 1a), in accordance with current guidelines (34).

Eighteen of the 45 IBS patients (54%) tested were diagnosed with BAM, of which 4 had severe BAM, 7 had moderate BAM and 7 had mild BAM. A further 5 patients were borderline BAM (16-20% retention). Using the 15% threshold, a positive BAM classification was reported in 40% of the IBS population tested. Mild BAM was identified in one control subject, who was subsequently diagnosed with IBS. As expected, a positive BAM diagnosis was more common in IBS-D (74%) than in IBS-M (35%) (p-value=0.03) (FIG. 1b).

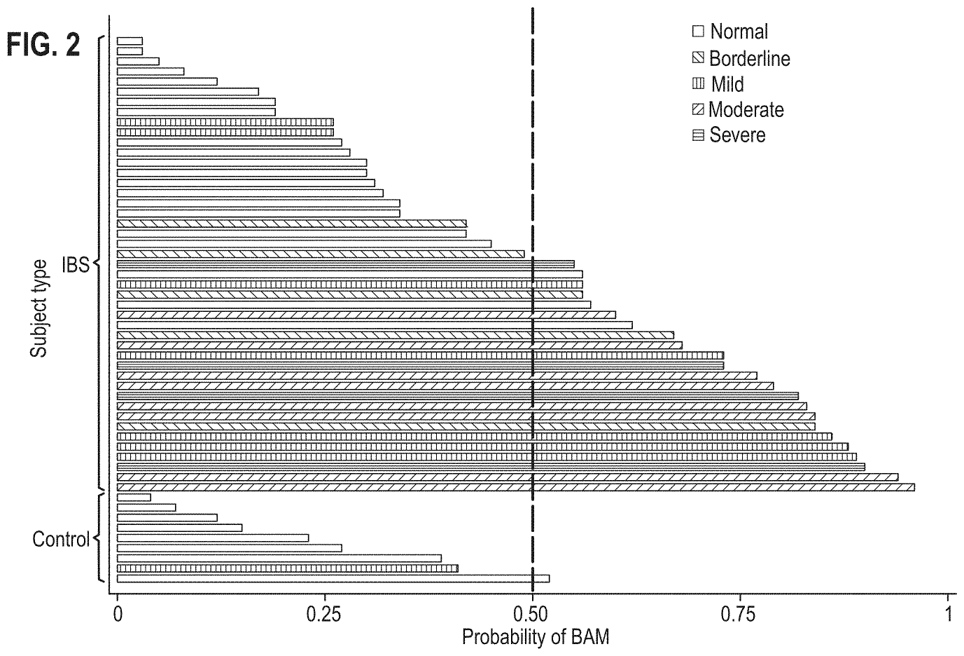

Only IBS patients in the severe BAM category showed a distinct separation in their microbiota from the microbiota of patients with normal, mild, moderate, or borderline BAM diagnoses (using analysis as set out, see FIG. 1c). To further investigate the biological impact of BAM, untargeted fecal metabolite analysis was performed by GC- and LC-MS (as set out above). The fecal metabolome of IBS patients with a severe BAM diagnosis was significantly different from that of the other BAM classes in the patients with IBS who had undergone the SeHCAT assay (FIG. 1d). Machine learning applied to fecal metabolome data successfully predicted BAM with an AUC of 0.92 for detecting all BAM classes (including borderline) in a test set of IBS patients and controls; the model performed with 100% accuracy (sensitivity: 0.80 and specificity: 0.86) for severe and moderate BAM, 62.5% for mild BAM and 60% for borderline BAM (FIG. 2 and Table 1). The main predictive metabolites for BAM included L-lysine, two glycerophospholipids and a bile acid (ursodeoxycholic acid (UDCA)). Elevated levels of these categories of compounds have been associated with altered fatty acid metabolism and disease (35), (36).

Machine learning applied to the microbiome OTU dataset identified BAM (AUC: 0.95, sensitivity: 0.88 and specificity: 0.93) (Table 2). While the metabolomics model performed with 100% accuracy for severe and moderate BAM, the OTU model resulted in fewer misclassifications (five) compared to the fecal metabolomics model (nine). There was no overlap in misclassified subjects between the models, indicating that a combined microbiome-metabolome model would increase BAM prediction accuracy.

Discussion

It has been shown that the subset of IBS-D and IBS-M patients that have severe bile acid malabsorption have an altered microbiome and fecal metabolome.

The microbiome among IBS clinical subtypes does not significantly differ, and the clinical utility of assigning patients to these categories is debatable. However, a subset of IBS-D and IBS-M patients with BAM were identified who were distinguishable by a microbiome and metabolomic signature. Others have reported altered microbiota in IBS-D but did not stratify for BAM (15). It is also noteworthy that transit time (reflected by Bristol Stool Score) is a major co-variate with microbiota composition (37) but the tendency for IBS patients to alternate between the mixed, constipation and diarrhea subtypes, may mask or ‘average out’ microbiota associations with transit time. Regardless, all three subtypes can be distinguished from controls by a common fecal microbiome signature.

BAM was detected by SeHCAT in over half of the combined IBS-D and M subjects tested. Differences in the microbiome were most evident in the severe BAM group. The unrecognized presence of appreciable numbers of subjects with BAM may have contributed to low treatment success rates compared to placebo in previous trials of various IBS therapeutics (38). While subjects in the severe BAM category had a significantly altered microbiome, a fecal metabolomics signature was identified for all BAM-diagnosed subjects. This fecal metabolomics signature for BAM will readily have clinical application as it requires instrumentation that is more convenient, more accessible and less expensive than SeHCAT (which is not currently available in the USA).

### Example 2—Fecal Metabolome Analysis of IBS Patients with Bile Acid Malabsorption (BAM) with an Alternative Machine Learning Pipeline

Materials and Methods

Subject recruitment: Eighty patients aged 16-70 years with IBS meeting the Rome IV criteria were recruited at Cork University Hospital. Clinical subtyping of the patients (21) was as follows: IBS with constipation (IBS-C), mixed IBS (IBS-M) or IBS with diarrhea (IBS-D). Sixty-five controls of the same age range and of the same ethnicity and geographic region were recruited. Descriptive statistics for the study population are presented in Table 4.

Exclusion criteria included the use of antibiotics within 6 weeks prior to study enrolment, other chronic illnesses including gastrointestinal diseases, severe psychiatric disease, abdominal surgery other than hernia repair or appendectomy. Standard-of-care blood analysis was carried out on all participants if recent results were not available, and all subjects were tested to exclude coeliac disease. The inclusion/exclusion criteria for the control population were the same as for the IBS population with the exception of having to fulfil the Rome IV criteria for IBS. Gastrointestinal (GI) symptom history, psychological symptoms, diet, medical history and medication data were collected on each participant (both IBS and controls) and using the following questionnaires: Bristol Stool Score (BSS), Hospital Anxiety and Depression Scale (HADS) (22); Food Frequency Questionnaire (FFQ) (23). IBS-D and IBS-M patients reporting diarrhoea as well as a subset of consenting control subjects were assessed for bile acid malabsorption by SeHCAT, a radiolabelled synthetic bile acid which is used to clinically diagnosis of BAM which is not metabolized by bacteria and passes through the enterohepatic circulation as endogenous bile acids. Ethical approval for the study was granted by the Cork Research Ethics Committee (protocol number: 4DC001) before commencing the study and all participants provided written informed consent to take part.

Sample collection: Fecal and urine samples were collected from all participants for metabolomics profiling. Subjects collected a freshly voided fecal sample at home using a collection kit and brought the sample to the clinic that day, when a fresh urine sample was collected. Samples were kept at 4° C. until brought to the laboratory for storage at −80° C. which was within a few hours of the sample collection.

Fecal GC/LC MS: 1 g samples of frozen feces were sent on dry ice to Metabolomic Discoveries (now Metabolon), Potsdam, Germany. For LC-MS, the samples were dried and resuspended to a final concentration of 10 mg per 400 μl before analysis. GC-MS and SCFA analysis were performed using wet samples. Untargeted metabolomics analysis was performed using liquid chromatography (LC) and Solid Phase Microextraction (SPME) gas chromatography (GC) and metabolites were identified using electrospray ionization mass spectrometry (ESI-MS). SCFA analysis was also performed by LC-tandem mass spectrometry.

Bioinformatics analysis of fecal metabolome data: Fecal MS metabolomics data was returned for all IBS subjects (n=80) and all but 2 controls (n=63) as these did not pass QC or no sample was available. 2,933 metabolites were returned from untargeted fecal metabolomics analysis carried out by the service provider of which 753 were identified. Metabolites identified using LC-MS were not normalized, since the fecal samples were already normalized with dry weight (10 mg per 400 μl) during sample preparation. Metabolites identified using GC-MS were normalized with corresponding sample wet weights. Only the identified metabolites were considered for further analyses. Machine learning analysis was carried out as described below. Summary statistics for all datasets were generated using the Wilcoxon rank sum test with q-value adjustment for multiple testing.

BAM SeHCAT assay: SeHCAT was administered at Cork University Hospital as a single capsule dose containing less than 0.1 mg of tauroselcholic acid (GE Healthcare, UK) and with a radioactivity dose of 370 kBq at the reference date. A baseline whole-body absorption reading using an uncollimated gamma counter (Siemens Ecam camera) was taken for each subject 2-3 hours after capsule administration. A follow-up scan was taken 7 days later and the proportion of bile acid retention was calculated; a value of <15% retention indicated mild to severe BAM with a SeHCAT score of 15-20% representing a borderline classification as discussed by Watson et al (2015) (31).

Machine learning: An in-house machine learning pipeline was applied to the fecal metabolomic data. The machine learning pipeline used in this example is similar to the machine learning pipeline used in Example 1, but comprised additional optimization and validation steps, using a two step approach within a ten-fold cross-validation. Within each validation fold Least Absolute Shrinkage and Selection Operator (LASSO) feature selection was carried out followed by Random Forest (RF) modelling and an optimised model was validated against the cross validation test data which is external to the cross-validation training subset.

The models were implemented using R software version 3.4.0, using package glmnet version 2.0-10 for LASSO feature selection, and RF package randomForest version 4.6-12.

The fecal metabolome sample profiles were log₁₀ transformed before they were analysed in the machine learning pipeline. Only IBS samples having SeHCAT information were transformed. Samples with borderline BAM were then removed, and the remaining samples classified as BAM (19 samples) or Normal (21 samples). The classified samples were then analysed in the machine learning pipeline.

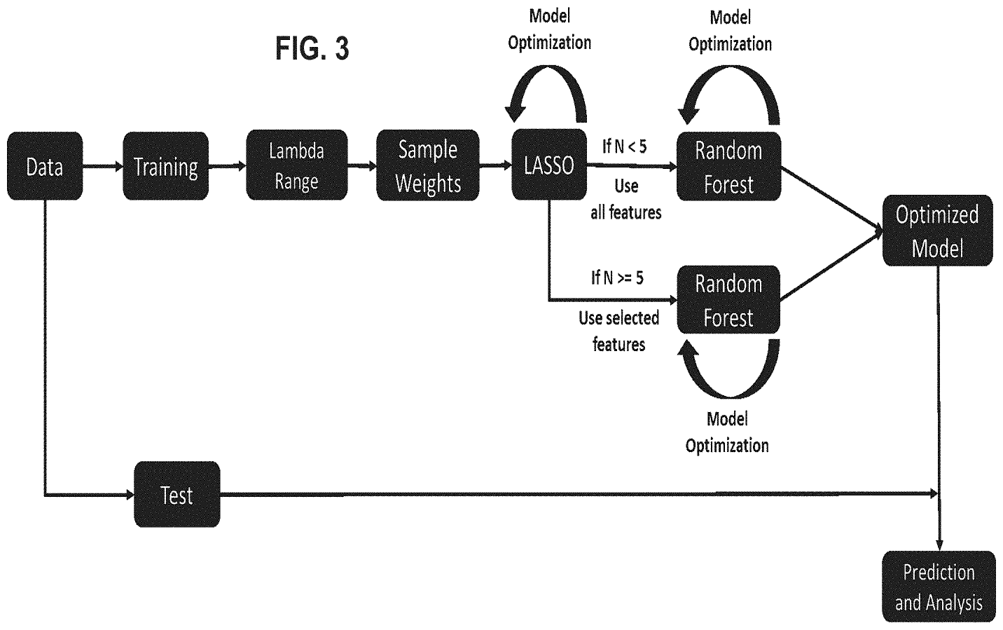

FIG. 3 shows the machine learning pipeline used in this example. The classified fecal metabolome sample profiles were first split into a training set and a test set. The training set was then used to generate an optimal lambda (λ) range for use by the LASSO algorithm. The optimal lambda (λ) range was generated using the previously described cross-validated LASSO and using the glmnet package (version 2.0-18). Pre-determination of an optimal lambda (λ) range reduces the computational time to run the pipeline and removes the need for a user to specify the ranges manually.

After determination of the lambda (λ) range, the samples were assigned weights based on their class probabilities. The weights assigned to the training samples in this step were used in all subsequent applicable steps.

A LASSO algorithm substantially as described in Example 1 was then applied to the weighted training samples. In this example, the LASSO algorithm used the previously calculated optimal lambda (λ) range, and used the Caret (version 6.0-84 in this example) and glmnet (version 2.0-18 in this example) packages, The ROC AUC (receiver operating characteristic, area under curve) metric was calculated using Leave-One-Out cross validation. Leave-One-Out cross validation was used to maximise the number of samples available for model optimization. The feature coefficients identified by the optimized LASSO algorithm were extracted and features with non-zero coefficients were selected for further analysis. In FIG. 3, N refers to the number of features returned by the LASSO algorithm. If the number of features selected by LASSO was fewer than 5, then all of the features (pre-LASSO) were used to generate the random forest, i.e. the LASSO filtering was ignored by the random forest generator. If the number of features selected by LASSO was greater than or equal to 5, then only those features selected by LASSO were used for generation of the random forest.

Following feature selection using LASSO, an optimized random forest classifier (with 1500 trees) was generated using the selected features. Random forest generation was performed using Caret (version 6.0-84) and internal cross validation, by tuning the ‘mtry’ parameter to maximise the ROC AUC metric. The optimized random forest classifier was then applied to the test set and the performance of the classifier was calculated via the AUC, sensitivity, and specificity metrics.

Results

Fecal Metabolome is Predictive of BAM Classes

Fecal metabolome profile was investigated for its predictive ability to classify samples as BAM or non-BAM. Cross-validation was Leave-One-Out CV. Leave-One-Out CV was used to ensure the maximal number of samples available for model optimization.

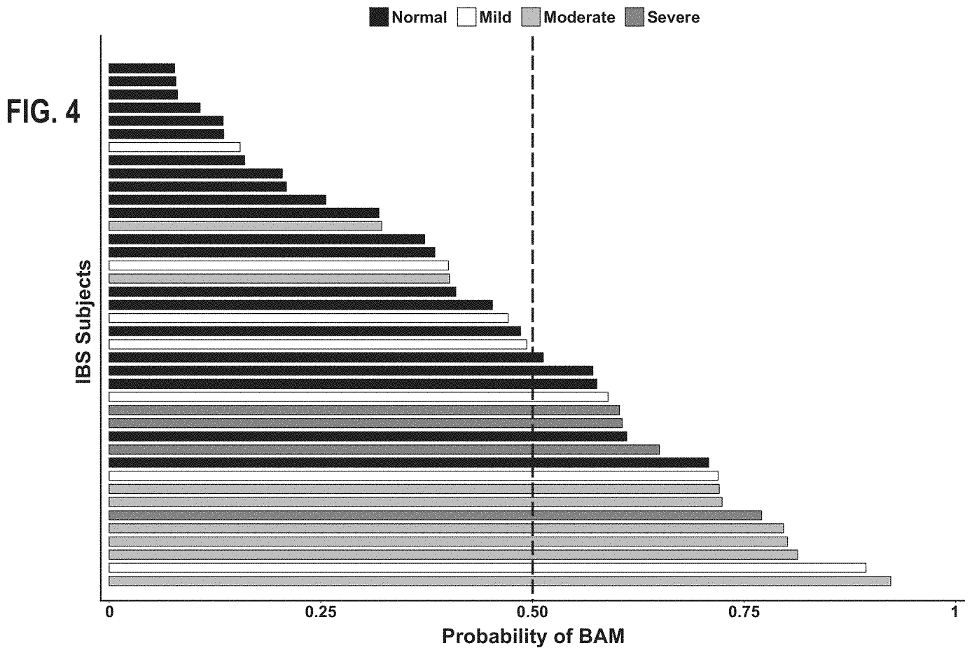

Machine learning to the fecal metabolome dataset of the IBS patients who underwent the SeHCAT assay, but excluding patients with a borderline BAM diagnosis. The predictive model successfully identified the subjects that had BAM with an AUC of 0.85 in all three BAM grades. The model performed with 100% accuracy for severe BAM, 75% for moderate BAM and 43% for mild BAM. The performance summary, and feature details are described in table 7 and shown in FIG. 4. Features selected by LASSO having coefficients less than zero are associated with BAM while positive coefficients are associated with Normal. The cross-validation results suggest that the fecal metabolome profile is predictive of BAM. The overall test performance was an AUC of 0.852, sensitivity of 0.684, specificity of 0.762, and accuracy of 0.725, with 11 misclassifications (Table 7).

The classification threshold was optimized to achieve maximum sensitivity and specificity using pROC package (version 1.15.0) and Youden J score. The obtained optimized values for Sensitivity and Specificity were 0.684, and 0.904, respectively.

The metabolites identified using this pipeline as predictive for BAM are listed in Table 7. Among the main predictive metabolites were a range of glycerophospholipids. Elevated levels of these compounds have been associated with altered fatty acid metabolism and disease. Among the main predictive metabolites for BAM were 1,3-di-(5Z,8Z,11Z,14Z,17Z-eicosapentaenoyl)-2-hydroxy-glycerol (d5) and dimethyl benzyl carbinyl butyrate.

Discussion

It has been shown via machine learning analysis that fecal metabolome is predictive of BAM status in IBS. It is shown that the subset of IBS-D and IBS-M patients with bile acid malabsorption have an altered fecal metabolome that can potentially be used to distinguish these subjects without requiring a SeHCAT test.

The microbiome among IBS clinical subtypes does not significantly differ, and the clinical utility of assigning patients to these categories is debatable. However, a subset of IBS-D and IBS-M patients with BAM were identified who were distinguishable by metabolomic signature. Others have reported altered microbiota in IBS-D but did not stratify for BAM (15). BAM was detected by SeHCAT in over half of the combined IBS-D and M subjects tested. Differences in the microbiome were most evident in the severe BAM group. The unrecognized presence of appreciable numbers of subjects with BAM may have contributed to low treatment success rates compared to placebo in previous trials of various IBS therapeutics (38). While subjects in the severe BAM category had a significantly altered microbiome, a fecal metabolomics signature was identified for all BAM-diagnosed subjects. This fecal metabolomics signature for BAM will readily have clinical application as it requires instrumentation that is more convenient, more accessible and less expensive than SeHCAT (which is not currently available in the USA).

The above described pipeline for recognising the fecal metabolomics signature of BAM will also have clinical application as it similarly utilises instrumentation that is more convenient, more accessible and less expensive than SeHCAT.

### CONCLUSION

The findings of the current study have clinical implications. A fecal metabolomic profile has been linked with BAM which can accurately distinguish it from non-BAM related IBS.

The taxa and metabolites that distinguish BAM subjects from non-BAM related IBS subjects identified here may be targeted by a range of microbiota-directed therapies such as fecal transplants, antibiotics, probiotics or live biotherapeutics.

### Tables

TABLE 1
FECAL METABOLOMICS MACHINE LEARNING LASSO AND RANDOM FOREST (RF) STATISTICS FOR BAM PREDICTION

| LASSO | | | | RF | | | |
|---|---|---|---|---|---|---|---|
| lambda | AUC | Sens | Spec | mtry | AUC | Sens | Spec |
| 0.100 | 0.880 | 0.680 | 0.862 | 1 | 0.923 | 0.800 | 0.862 |
| Leave-One-Out Cross Validation | | | | Leave-One-Out Cross Validation | | | |
| Reference | | | | Reference | | | |
| Prediction | BAM | | Normal | Prediction | BAM | | Normal |
| BAM | 17 | | 4 | BAM | 20 | | 4 |
| Normal | 8 | | 25 | Normal | 5 | | 25 |
| Accuracy | 0.78 | | | Accuracy | 0.83 | | |
| | | | | | | | Median |

| Rank # | Ranking | Metabolite | Rank # | Ranking | Metabolite | Abundance |
|---|---|---|---|---|---|---|
| 1 | 100.00 | PG(P-16:0/14:0) | 1 | 100 | PG(P-16:0/14:0) | 548624.65 |
| 2 | 49.31 | 2-Ethylsuberic acid | 2 | 89.63 | 2-Ethylsuberic acid | 515705.82 |
| 3 | 30.22 | Glu-Glu-Gly-Tyr | 3 | 87.54 | Glu-Glu-Gly-Tyr | 255312.83 |
| 4 | 29.44 | 1,2,3-Tris(1- | 4 | 81.21 | 1,2,3-Tris(1- | 555486.83 |
| | | ethoxyethoxy)propane | | | ethoxyethoxy)propane | |
| 5 | 15.30 | PG(O-30:1) | 5 | 73.68 | PG(O-30:1) | 184213.09 |
| 6 | 8.43 | Ursodeoxycholic acid | 6 | 55.81 | Ursodeoxycholic acid | 9239005.5 |
| 7 | 3.74 | MG(22:2(13Z,16Z)/0:0/0:0) | 7 | 37.65 | MG(22:2(13Z,16Z)/0:0/0:0) | 96634.95 |
| 8 | 3.62 | L-Lysine | 8 | 20.70 | L-Lysine | 325085.95 |
| 9 | 2.68 | O-Phosphoethanolamine | 9 | 12.39 | O-Phosphoethanolamine | 165434.53 |
| 10 | 0.36 | PE(22:5(7Z,10Z, | 10 | 4.13 | PE(22:5(7Z,10Z, | 64169.24 |
| | | 13Z,16Z,19Z)/24:0) | | | 13Z,16Z,19Z)/24:0) | |
| 11 | 0.07 | Heptadecanoic acid | 11 | 0 | Heptadecanoic acid | 568540.11 |

Analysis had 2 classes: BAM and Normal (Non-BAM) and included fecal metabolomics data from 54 subjects (IBS n = 45; Control n = 9) tested for BAM
IBS-BAM (n = 24) and Control-BAM (n = 1)
753 predictors were used in the model
All BAM classes from borderline to severe were included in the BAM group.

TABLE 2
16S OTU Machine learning LASSO and Random Forest (RF) statistics for BAM prediction

| LASSO | | | | RF | | | |
|---|---|---|---|---|---|---|---|
| lambda | AUC | Sens | Spec | mtry | AUC | Sens | Spec |
| 0.08 | 0.48 | 0.44 | 0.62 | 1 | 0.95 | 0.88 | 0.93 |

| Leave-One-Out Cross Validation | | | | Leave-One-Out Cross Validation | | |
|---|---|---|---|---|---|---|
| Reference | | | | Reference | | |
| Prediction | BAM | Normal | | Prediction | BAM | Normal |
| BAM | 11 | 11 | | BAM | 22 | 2 |
| Normal | 14 | 18 | | Normal | 3 | 27 |
| Accuracy | 0.54 | | | Accuracy | 0.91 | |
| RF Ranking | | | | | | |
| Rank # | Ranking | Phylum | Class | Order | Family | Genus |
| 1 | 100.00 | Firmicutes | Clostridia | Clostridiales | Lachnospiraceae | Blautia |
| 2 | 98.77 | Bacteroidetes | Bacteroidia | Bacteroidales | Bacteroidaceae | Bacteroides |
| 3 | 98.52 | Firmicutes | Clostridia | Clostridiales | | |
| 4 | 97.79 | Firmicutes | Clostridia | Clostridiales | Ruminococcaceae | Faecalibacterium |
| 5 | 94.88 | Firmicutes | Clostridia | Clostridiales | Ruminococcaceae | Oscillibacter |
| | | | | | | Lachnospiracea_incertae_sedis |
| 6 | 91.42 | Firmicutes | Clostridia | Clostridiales | Lachnospiraceae | |
| 7 | 89.06 | Firmicutes | Clostridia | Clostridiales | Lachnospiraceae | |
| 8 | 85.01 | Firmicutes | Clostridia | Clostridiales | Lachnospiraceae | |
| 9 | 77.62 | Firmicutes | Clostridia | Clostridiales | Ruminococcaceae | |
| 10 | 76.30 | Firmicutes | Clostridia | Clostridiales | Lachnospiraceae | Ruminococcus2 |
| 11 | 68.45 | Firmicutes | Clostridia | Clostridiales | Lachnospiraceae | |
| 12 | 68.05 | Actinobacteria | Actinobacteria | Bifidobacteriales | Bifidobacteriaceae | Bifidobacterium |
| 13 | 65.07 | Firmicutes | Clostridia | Clostridiales | Lachnospiraceae | Coprococcus |
| 14 | 55.32 | Firmicutes | Clostridia | Clostridiales | | |
| 15 | 51.76 | Bacteroidetes | Bacteroidia | Bacteroidales | Prevotellaceae | Paraprevotella |
| 16 | 50.80 | Bacteroidetes | Bacteroidia | Bacteroidales | Bacteroidaceae | Bacteroides |
| 17 | 50.36 | Firmicutes | Clostridia | Clostridiales | Ruminococcaceae | |
| 18 | 49.12 | Firmicutes | Clostridia | Clostridiales | Ruminococcaceae | Gemmiger |
| 19 | 45.13 | Firmicutes | Clostridia | Clostridiales | Ruminococcaceae | |
| | | | | Selenomonadales | | |
| 20 | 38.95 | Firmicutes | Negativicutes | | Veillonellaceae | Dialister |
| 21 | 35.45 | Firmicutes | Clostridia | Clostridiales | | |
| 22 | 34.52 | Firmicutes | Clostridia | Clostridiales | | |
| 23 | 33.75 | Firmicutes | Clostridia | Clostridiales | | |
| 24 | 22.41 | Firmicutes | Clostridia | Clostridiales | Lachnospiraceae | |
| 25 | 19.51 | Firmicutes | Clostridia | Clostridiales | Ruminococcaceae | Faecalibacterium |
| 26 | 3.64 | Firmicutes | Clostridia | Clostridiales | | |
| | | | | Selenomonadales | | |
| 27 | 1.48 | Firmicutes | Negativicutes | | Veillonellaceae | Megamonas |
| 28 | 0.00 | Actinobacteria | Actinobacteria | Coriobacteriales | Coriobacteriaceae | |

Analysis had 2 classes: BAM and Normal (Non-BAM) and included fecal metabolomics data from 54 subjects (IBS n = 45; Control n = 9)
tested for BAM
IBS-BAM (n = 24) and Control-BAM (n = 1)
1754 predictors were used in the model
All BAM classes from borderline to severe were included in the BAM group
Taxonomy classified using the RDP classfier, database version 2.10.1.

TABLE 3
16S OTU Machine learning LASSO and Random Forest (RF) statistics
for BAM prediction sequence information

| Rank # | Ranking | Phylum | Class | Order | Family |
|---|---|---|---|---|---|
| 1 | 100 | Firmicutes | Clostridia | Clostridiales | Lachnospiraceae |
| 2 | 98.77 | Bacteroidetes | Bacteroidia | Bacteroidales | Bacteroidaceae |
| 3 | 98.52 | Firmicutes | Clostridia | Clostridiales | |
| 4 | 97.79 | Firmicutes | Clostridia | Clostridiales | Ruminococcaceae |
| 5 | 94.88 | Firmicutes | Clostridia | Clostridiales | Ruminococcaceae |
| 6 | 91.42 | Firmicutes | Clostridia | Clostridiales | Lachnospiraceae |
| 7 | 89.06 | Firmicutes | Clostridia | Clostridiales | Lachnospiraceae |
| 8 | 85.01 | Firmicutes | Clostridia | Clostridiales | Lachnospiraceae |
| 9 | 77.62 | Firmicutes | Clostridia | Clostridiales | Ruminococcaceae |
| 10 | 76.3 | Firmicutes | Clostridia | Clostridiales | Lachnospiraceae |
| 11 | 68.45 | Firmicutes | Clostridia | Clostridiales | Lachnospiraceae |
| 12 | 68.05 | Actinobacteria | Actinobacteria | Bifidobacteriales | Bifidobacteriaceae |
| 13 | 65.07 | Firmicutes | Clostridia | Clostridiales | Lachnospiraceae |
| 14 | 55.32 | Firmicutes | Clostridia | Clostridiales | |
| 15 | 51.76 | Bacteroidetes | Bacteroidia | Bacteroidales | Prevotellaceae |
| 16 | 50.8 | Bacteroidetes | Bacteroidia | Bacteroidales | Bacteroidaceae |
| 17 | 50.36 | Firmicutes | Clostridia | Clostridiales | Ruminococcaceae |
| 18 | 49.12 | Firmicutes | Clostridia | Clostridiales | Ruminococcaceae |
| 19 | 45.13 | Firmicutes | Clostridia | Clostridiales | Ruminococcaceae |
| 20 | 38.95 | Firmicutes | Negativicutes | Selenomonadales | Veillonellaceae |
| 21 | 35.45 | Firmicutes | Clostridia | Clostridiales | |
| 22 | 34.52 | Firmicutes | Clostridia | Clostridiales | |
| 23 | 33.75 | Firmicutes | Clostridia | Clostridiales | |
| 24 | 22.41 | Firmicutes | Clostridia | Clostridiales | Lachnospiraceae |
| 25 | 19.51 | Firmicutes | Clostridia | Clostridiales | Ruminococcaceae |
| 26 | 3.64 | Firmicutes | Clostridia | Clostridiales | |
| 27 | 1.48 | Firmicutes | Negativicutes | Selenomonadales | Veillonellaceae |
| 28 | 0 | Actinobacteria | Actinobacteria | Coriobacteriales | Coriobacteriaceae |

| Rank # | Genus | Sequence |
|---|---|---|
| 1 | Blautia | Cctacgggtggcagcagtggggaatattgcacaatggggga |
| | | aaccctgatgcagcgacgccgcgtgaaggaagaagtatctc |
| | | ggtatgtaaacttctatcagcagggaagatagtgacggtacct |
| | | gactaagaagccccggctaactacgtgccagcagccgcggt |
| | | aatacgtagggggcaagcgttatccggatttactgggtgtaaa |
| | | gggagcgtagacggactggcaagtctgatgtgaaaggcgg |
| | | gggctcaacccctggactgcattggaaactgttagtcttgagtg |
| | | ccggagaggtaagcggaattcctagtgtagcggtgaaatgcg |
| | | tagatattaggaggaacaccagtggcgaaggcggcttactgg |
| | | acggtaactgacgttgaggctcgaaagcgtggggagcaaac |
| | | aggattagataccctggtagtc (SEQ ID No: 1) |
| 2 | Bacteroides | Cctacggggggctgcagtgaggaatattggtcaatgggcgat |
| | | ggcctgaaccagccaagtagcgtgaaggatgactgccctat |
| | | gggttgtaaacttcttttataaaggaataaagtcgggtatgcata |
| | | cccgtttgcatgtactttatgaataaggatcggctaactccgtgc |
| | | cagcagccgcggtaatacggaggatccgagcgttatccgga |
| | | tttattgggtttaaagggagcgtagatggatgtttaagtcagttgt |
| | | gaaagtttgcggctcaaccgtaaaattgcagttgatactggatg |
| | | tcttgagtgcagttgaggcaggcggaattcgtggtgtagcggt |
| | | gaaatgcttagatatcacgaagaactccgattgcgaaggcag |
| | | cctgctaagctgcaactgacattgaggctcgaaagtgtgggta |
| | | tcaaacaggattagataccccagtagtc (SEQ ID No: 2) |
| 3 | | Cctacggggggctgcagtggggaatattgcacaatgggcga |
| | | aagcctgatgcagcaacgccgcgtgagcgaagaaggtcttc |
| | | ggatcgtaaagctctgtccttggggaagataatgacggtaccc |
| | | ttggaggaagccccggctaactacgtgccagcagccgcggt |
| | | aatacgtagggggcaagcgttatccggaattattgggcgtaa |
| | | agagtgcgtaggtggttacctaagcagggggtgaaaggcac |
| | | tggcttaaccaatgtcagccccctgaactgggtaccttgagtgc |
| | | aggagaggaaagcggaattcctagtgtagcggtgaaatgcg |
| | | tagatattaggaggaacaccagtggcgaaggcggctttctgg |
| | | actgttactgacactgaggcacgaaagtgtggggagcaaac |
| | | aggattagataccccagtagtc (SEQ ID No: 3) |
| 4 | Faecalibacterium | Cctacggggggctgcagtggggaatattgcacaatggggga |
| | | aaccctgatgcagcgacgccgcgtggaggaagaaggtcttc |
| | | ggattgtaaactcctgttgttgaggaagataatgacggtactca |
| | | acaaggaagtgacggctaactacgtgccagcagccgcggt |
| | | aaaacgtaggtcacaagcgttgtccggaattactgggtgtaaa |
| | | gggagcgcaggcgggaagacaagttggaagtgaaatccat |
| | | gggctcaacccatgaactgctttcaaaactgtttttcttgagtagt |
| | | gcagaggtaggcggaattcccggtgtagcggtggaatgcgta |
| | | gatatcgggaggaacaccagtggcgaaggcggcctactgg |
| | | gcaccaactgacgctgaggctcgaaagtgtgggtagcaaac |
| | | aggattagataccccagtagtc (SEQ ID No: 4) |
| 5 | Oscillibacter | Cctacggggggctgcagtggggaatattgggcaatggacgc |
| | | aagtctgacccagcaacgccgcgtgaaggaagaaggctttc |
| | | gggttgtaaacttcttttgtcagggaacagtagaagagggtac |
| | | ctgacgaataagccacggctaactacgtgccagcagccgcg |
| | | gtaatacgtaggtggcaagcgttgtccggatttactgggtgtaa |
| | | agggcgtgcagccgggctggcaagtcaggcgtgaaatccc |
| | | agggctcaaccctggaactgcgtttgaaactgctggtcttgagt |
| | | accggagaggtcatcggaattccttgtgtagcggtgaaatgcg |
| | | tagatataaggaagaacaccagtggcgaaggcggatgact |
| | | ggacggcaactgacggtgaggcgcgaaagcgtggggagc |
| | | aaacaggattagataccccggtagtc (SEQ ID No: 5) |
| 6 | Lachnospiracea_ | Cctacggggggctgcagtggggaatattgcacaatggagga |
| | incertae_sedis | aactctgatgcagcgacgccgcgtgagtgaagaagtaattcg |
| | | ttatgtaaagctctatcagcagggaagatagtgacggtacctg |
| | | actaagaagctccggctaaatacgtgccagcagccgcggta |
| | | atacgtatggagcaagcgttatccggatttactgggtgtaaag |
| | | ggagtgtaggtggccatgcaagtcagaagtgaaaatccggg |
| | | gctcaaccccggaactgcttttgaaactgtaaggctggagtgc |
| | | aggaggggtgagtggaattcctagtgtagcggtgaaatgcgt |
| | | agatattaggaggaacaccagtggcgaaggcggctcactgg |
| | | actgtaactgacactgaggctcgaaagcgtggggagcaaac |
| | | aggattagataccccagtagtc (SEQ ID No: 6) |
| 7 | | Cctacggggggcagcagtggggaatattgcacaatggggg |
| | | aaaccctgatgcagcgacgccgcgtgaaggaagaagtattt |
| | | cggtatgtaaacttctatcagcagggaagaaaatgacggtac |
| | | ctgactaagaagccccggctaactacgtgccagcagccgcg |
| | | gtaatacgtagggggcaagcgttatccggatttactgggtgta |
| | | aagggagcgtaggcggtctgacaagtcagaagtgaaagcc |
| | | cggggctcaactccgggactgcttttgaaactgccggactaga |
| | | ttgcaggagaggtaagtggaattcctagtgtagcggtgaaatg |
| | | cgtagatattaggaggaacaccagtggcgaaggcggcttact |
| | | ggactgtaaatgacgctgaggctcgaaagcgtggggagcaa |
| | | acaggattagatacccgtgtagtc (SEQ ID No: 7) |
| 8 | | Cctacgggtggctgcagtggggaatattgcacaatggggga |
| | | aaccctgatgcagcaacgccgcgtgagtgaagaagtatttcg |
| | | gtatgtaaagctctatcagcaggaaagaaaatgacggtacct |
| | | gactaagaagccccggctaactacgtgccagcagccgcggt |
| | | aatacgtagggggcaagcgttatccggatttactgggtgtaaa |
| | | gggagcgtagacggtgaggcaagtctgaagtgaaatgccg |
| | | gggctcaaccccggaactgctttggaaactgtcgtactagagt |
| | | gtcggaggggtaagcggaattcctagtgtagcggtgaaatgc |
| | | gtagatattaggaggaacaccagtggcgaaggcggcttgctg |
| | | gactgtaactgacactgaggctcgaaagcgtggggagcaaa |
| | | caggattagatacccttgtagtc (SEQ ID No: 8) |
| 9 | | Cctacggggggctgcagtggggaatattgcacaatggagga |
| | | aactctgatgcagcgacgccgcgtgagggaagaaggtcttc |
| | | ggattgtaaacctctgttgtcagggacgatgatgacggtacctg |
| | | acgaggaagccacggctaactacgtgccagcagccgcggt |
| | | aaaacgtaggtggcaagcgttgtccggaattactgggtgtaa |
| | | agggagcgcaggcgggagagcaagttgggagtgaaatctg |
| | | tgggctcaacccacaaattgctttcaaaactgtttttcttgagtgg |
| | | tgtagaggtaggcggaattcccggtgtagcggtggaatgcgt |
| | | agatatcgggaggaacaccagtggcgaaggcggcctactg |
| | | ggcactaactgacgctgaggctcgaaagcatgggtagcaaa |
| | | caggattagataccccggtagtc (SEQ ID No: 9) |
| 10 | Ruminococcus2 | Cctacggggggctgcagtggggaatattgcacaatggggga |
| | | aaccctgatgcagcgacgccgcgtgagcgaagaagtatttc |
| | | ggtatgtaaagctctatcagcagggaagaaaatgacggtacc |
| | | tgactaagaagccccggctaactacgtgccagcagccgcgg |
| | | taatacgtagggggcaagcgttatccggatttactgggtgtaa |
| | | agggagcgtagacggagcagcaagtctgatgtgaaaaccc |
| | | ggggctcaaccccgggactgcattggaaactgttgatctgga |
| | | gtgccggagaggtaagcggaattcctagtgtagcggtgaaat |
| | | gcgtagatattaggaagaacaccagtggcgaaggcggcttg |
| | | ctggacagtaactgacgttcaggctcgaaagcgtggggagc |
| | | aaacaggattagatacccttgtagtc (SEQ ID No: 10) |
| 11 | | Cctacgggtggcagcagtggggaatattgcacaatggggga |
| | | aaccctgatgcagcaacgccgcgtgagtgaagaagtatttcg |
| | | gtatgtaaagctctatcagcagggaagaaaatgacggtacct |
| | | gactaagaagccccggctaactacgtgccagcagccgcggt |
| | | aatacgtagggggcaagcgttatccggatttactgggtgtaaa |
| | | gggagcgcaggcggtacggcaagtcagatgtgaaaacccg |
| | | gggctcaaccccgggactgcatttgaaactgtcggactagag |
| | | tgccggagaggtaagtggaattcctagtgtagcggtgaaatg |
| | | cgtagatattaggaggaacaccagtggcgaaggcggcttact |
| | | aaaccataactgacactgaagcacgaaagcgtggggagca |
| | | aacaggattagatacccgggtagtc (SEQ ID No: 11) |
| 12 | Bifidobacterium | Cctacggggggctgcagtggggaatattgcacaatgggcgc |
| | | aagcctgatgcagcgacgccgcgtgagggatggaggccttc |
| | | gggttgtaaacctcttttatcggggagcaagcgagagtgagttt |
| | | acccgttgaataagcaccggctaactacgtgccagcagccg |
| | | cggtaatacgtagggtgcaagcgttatccggaattattgggcgt |
| | | aaagggctcgtaggcggttcgtcgcgtccggtgtgaaagtcc |
| | | atcgcttaacggtggatccgcgccgggtacgggcgggcttga |
| | | gtgcggtaggggagactggaattcccggtgtaacggtggaat |
| | | gtgtagatatcgggaagaacaccaatggcgaaggcaggtct |
| | | ctgggccgttactgacgctgaggagcgaaagcgtggggagc |
| | | gaacaggattagataccccagtagtc (SEQ ID No: 12) |
| 13 | Coprococcus | Cctacggggggcagcagtggggaatattgcacaatggggg |
| | | aaaccctgatgcagcgacgccgcgtgagcgaagaagtattt |
| | | cggtatgtaaagctctatcagcagggaagataatgacggtac |
| | | ctgactaagaagcaccggctaaatacgtgccagcagccgcg |
| | | gtaatacgtatggtgcaagcgttatccggatttactgggtgtaa |
| | | agggtgcgtaggtggtgagacaagtctgaagtgaaaatccg |
| | | gggcttaaccccggaactgctttggaaactgcctgactagagt |
| | | acaggagaggtaagtggaattcctagtgtagcggtgaaatgc |
| | | gtagatattaggaggaacaccagtggcgaaggcgacttactg |
| | | gactgctactgacactgaggcacgaaagcgtggggagcaa |
| | | acaggattagataccctggtagtc (SEQ ID No: 13) |
| 14 | | Cctacggggggcagcagtcgggaatattgcgcaatggagg |
| | | aaactctgacgcagtgacgccgcgtataggaagaaggttttc |
| | | ggattgtaaactattgtcgttagggaagatacaagacagtacc |
| | | taaggaggaagctccggctaactacgtgccagcagccgcgg |
| | | taatacgtagggagcaagcgttatccggatttattgggtgtaaa |
| | | gggtgcgtagacgggacaacaagttagttgtgaaatccctcg |
| | | gcttaactgaggaactgcaactaaaactattgttcttgagtgttg |
| | | gagaggaaagtggaattcctagtgtagcggtgaaatgcgtag |
| | | atattaggaggaacaccggtggcgaaggcgactttctggaca |
| | | ataactgacgttgaggcacgaaagtgtggggagcaaacag |
| | | gattagataccccagtagtc (SEQ ID No: 14) |
| 15 | Paraprevotella | Cctacggggggcagcagtgaggaatattggtcaatgggcgg |
| | | gagcctgaaccagccaagtagcgtgaaggacgacggccct |
| | | acgggttgtaaacttcttttataagggaataaagtgcgttacgtg |
| | | taatgttttgtatgtaccttatgaataagcatcggctaattccgtgc |
| | | cagcagccgcggtaatacggaagatgcgagcgttatccgga |
| | | tttattgggtttaaagggagcgtaggcgggcttttaagtcagcg |
| | | gtcaaatgtcacggctcaaccgtggccagccgttgaaactgc |
| | | aagccttgagtctgcacagggcacatggaattcgtggtgtagc |
| | | ggtgaaatgcttagatatcacgaagaactccgatcgcgaagg |
| | | cattgtgccggggcagcactgacgctgaggctcgaaagtgc |
| | | gggtatcaaacaggattagatacccctgtagtc (SEQ ID No: 15) |
| 16 | Bacteroides | Cctacgggaggcagcagtgaggaatattggtcaatgggcga |
| | | tggcctgaaccagccaagtagcgtgaaggatgactgccctat |
| | | gggttgtaaacttcttttataaaggaataaagtcgggtatgcata |
| | | cccgtttgtatgtaccttatgaataaggatcggctaactccgtgc |
| | | cagcagccgcggtaatacggaggatccgagcgttatccgga |
| | | tttattgggtttaaagggagcgtaggcggactattaagtcagct |
| | | gtgaaagtttgcggctcaaccgtaaaattgcagttgatactggt |
| | | cgtcttgagtgcagtagaggtaggcggaattcgtggtgtagcg |
| | | gtgaaatgcttagatatcacgaagaactccgattgcgaaggc |
| | | agcctgctaagctgcaactgacattgaggctcgaaagtgtgg |
| | | gtatcaaacaggattagatacccgagtagtc (SEQ ID No: 16) |
| 17 | | Cctacggggggctgcagtgggggatattgcacaatggggga |
| | | aaccctgatgcagcgacgccgcgtggaggaagaaggttttc |
| | | ggattgtaaactcctgtcgttagggacgataatgacggtaccta |
| | | acaagaaagcaccggctaactacgtgccagcagccgcggt |
| | | aaaacgtagggtgcaagcgttatccggatttactgggtgtaaa |
| | | gggagcgcaggcgggactgcaagttggatgtgaaataccgt |
| | | ggcttaaccacggaactgcatccaaaactgtagttcttgagtg |
| | | aagtagaggcaagcggaattccgagtgtagcggtgaaatgc |
| | | gtagagatggggaggaacaccagtggcgaaggcggcctgc |
| | | tgggctttaactgacgctgaggcacgaaagcgtgggtagcaa |
| | | acaggattagataccccagtagtc (SEQ ID No: 17) |
| 18 | Gemmiger | Cctacgggaggcagcagtgggggatattgcacaatggggg |
| | | aaaccctgatgcagcgacgccgcgtggaggaagaaggtttt |
| | | cggattgtaaactcctgtcgttagggacgataatgacggtacct |
| | | aacaagaaagcaccggctaactacgtgccagcagccgcgg |
| | | taaaacgtagggtgcaagcgttgtccggaattactgggtgtaa |
| | | agggagcgcagacggcactgcaagtctgaagtgaaagccc |
| | | ggggctcaaccccggtactgcattggaaactgtcgtactaga |
| | | gtgtcggaggggtaagcggaattcctagtgtagcggtgaaat |
| | | gcgtagatatcgggaggaacaccagtggcgaaggcgacct |
| | | actgggcaccaactgacgctgaggctcgaaagcatgggtag |
| | | caaacaggattagatacccctgtagtt (SEQ ID No: 18) |
| 19 | | Cctacggggggctgcagtggggaatattaggcaatgggcga |
| | | aagcctgacctagcgacgccgcgtgagggaagacggtcttc |
| | | ggattgtaaacctctgtcttcagggacgaagaagatgacggta |
| | | cctgaagaggaagccacggctaactacgtgccagcagccg |
| | | cggtaatacgtaggtggcgagcgttgtccggaattactgggtgt |
| | | aaagggagcgtaggcgggtacgcaagttgaatgtgaaaact |
| | | aacggctcaaccgatagttgcgttcaaaactgcggatcttgag |
| | | tgaagtagaggcaggcggaattcctagtgtagcggtaaaatg |
| | | cgtagatattaggaggaacaccagtggcgaaggcggcctgc |
| | | tgggctttaactgacgctgaggctcgaaagtgtggggagcaa |
| | | acaggattagataccccggtagtc (SEQ ID No: 19) |
| 20 | Dialister | Cctacggggggctgcagtggggaatcttccgcaatgggcga |
| | | aagcctgacggagcaacgccgcgtgagtgatgacggccttc |
| | | gggttgtaaaactctgtgatccgggacgaaaaggcagagtgc |
| | | gaagaacaaactgcattgacggtaccggaaaagcaagcca |
| | | cggctaactacgtgccagcagccgcggtaatacgtaggtgac |
| | | aagcgttgtccggatttactgggtgtaaagggcgcgtaggcgg |
| | | actgtcaagtcagtcgtgaaataccggggcttaaccccgggg |
| | | ctgcgattgaaactgacagccttgagtatcggagaggaaagt |
| | | ggaattcctagtgtagcggtgaaatgcgtagagattaggaag |
| | | aacaccggtggcgaaggcgactttctggacgaaaactgacg |
| | | ctgaggcgcgaaagcgtggggagcaaacaggattagatac |
| | | ccgggtagtc (SEQ ID No: 20) |
| 21 | | Cctacgggtggctgcagtgggggatattgcacaatggaggg |
| | | aactctgatgcagcaacgccgcgtgaaggacgaaggccttc |
| | | gggttgtaaacttctgtccttggtgacgaagaaagtgacggta |
| | | gccagggaggaagccacggctaactacgtgccagcagccg |
| | | cggtaatacgtaggtggcgagcgttgtccggaattactgggtgt |
| | | aaagggtgcgtaggcggcttctaaagtcagatgtgaaatacc |
| | | gcagctcaactgcggggctgcatttgaaacttgggagcttgag |
| | | tgaagtagaggtaagcggaattcctagtgtagcggtggaatg |
| | | cgtagatattaggaggaacaccagtggcgaaggcggcttact |
| | | gggctttaactgacgctgaggcacgaaagcgtggggagcaa |
| | | acaggattagataccccagtagtc (SEQ ID No: 21) |
| 22 | | Cctacgggaggctgcagtggggaatattgggcaatgggcga |
| | | aagcctgacccagcaacgccgcgtgaaggaagaaggcctt |
| | | cgggttgtaaacttcttttaagagggacgaagaagtgacggta |
| | | cctcttgaataagccacggctaactacgtgccagcagccgcg |
| | | gtaatacgtaggtggcaagcgttgtccggatttattgggtgtaa |
| | | agggagcgcagacggcactgcaagtctgaagtgaaagccc |
| | | ggggctcaaccccgggactgctttggaaactgtagagctaga |
| | | gtgctggagaggcaagcggaattcctagtgtagcggtgaaat |
| | | gcgtagatattaggaggaacaccagtggcgaaggcggctta |
| | | ctggacggtaactgacgttgaggctcgaaagcgtggggagc |
| | | aaacaggattagatacccgtgtagc (SEQ ID No: 22) |
| 23 | | Cctacgggtggctgcagtgggggatattgcgcaatgggggc |
| | | aaccctgacgcagcaacgccgcgtgaaggaagaaggctttc |
| | | gggttgtaaacttcttttgtcggggacgaaacaaatgacggtac |
| | | ccgacgaataagccacggctaactacgtgccagcagccgc |
| | | ggtaatacgtagggggctagcgttatccggaattactgggcgt |
| | | aaagggtgcgtaggtggtttcttaagtcagaggtgaaaggcta |
| | | cggctcaaccgtagtaagcctttgaaactgggaaacttgagtg |
| | | caggagaggagagtggaattcctagtgtagcggtgaaatgc |
| | | gtagatattaggaggaacaccagttgcgaaggcggctctctg |
| | | gactgtaactgacactgaggcacgaaagcgtggggagcaa |
| | | acaggattagataccctagtagtc (SEQ ID No: 23) |
| 24 | | Cctacgggaggcagcagtggggaatattgcacaatggggg |
| | | aaaccctgatgcagcgacgccgcgtgaaggatgaagtatttc |
| | | ggtatgtaaagctctatcagtagggaagaaaatgacggtacc |
| | | tgactaagaagcaccggctaaatacgtgccagcagccgcgg |
| | | taatacgtatggtgcaagcgttatccggatttactgggtgtaaa |
| | | ggaagtgtaggtggccaggcaagtcagaagtgaaagcccg |
| | | gggctcaaccccgggactgcttttgaaactgcagggctagag |
| | | tgcaggagaggtaagtggaattcctagtgtagcggtgaaatg |
| | | cgtagatattaggaggaacaccagtggcgaaggcggcttgct |
| | | ggacgatgactgacgttgaggctcgaaagcgtggggagcaa |
| | | acaggattagataccctagtagtc (SEQ ID No: 24) |
| 25 | Faecalibacterium | Cctacggggggctgcagtgagggatattgggcaatggggga |
| | | aaccctgacccagcgacgccgcgtgagggaagacggtcttc |
| | | ggattgtaaacctctgtctttggggacgaaaaaggacggtacc |
| | | caaggaggaagctccggctaactacgtgccagcagccgcg |
| | | gtaatacgtagggagcgagcgttgtccggaattactgggtgta |
| | | aagggagcgcaggcgggaaggcaagttggaagtgaaatc |
| | | catgggctcaacccatgaactgctttcaaaactgtttttcttgagt |
| | | agtgcagaggtaggcggaattcccggtgtagcggtggaatgc |
| | | gtagatattcggaggaacaccagtggcgaaggcggcctact |
| | | gggctttaactgacgctgaggctcgaaagtgtggggagcaaa |
| | | caggattagataccccggtagtc (SEQ ID No: 25) |
| 26 | | Cctacgggaggctgcagtggggaatattgcacaatggggga |
| | | aaccctgatgcagcaacgccgcgtgaaggatgacggttttcg |
| | | gattgtaaacttcttttcttagtgacgaagacagtgacggtagct |
| | | aaggaataagcatcggctaactacgtgccagcagccgcggt |
| | | aatacgtaggatgcaagcgttatccggatttactgggtgtaaa |
| | | gggagcgtaggtggcgaggcaagccagaagtgaaaaccc |
| | | ggggctcaaccgcgggattgcttttggaactgtcatgctagagt |
| | | gcaggaggggtgagcggaattcctagtgtagcggtgaaatg |
| | | cgtagatattaggaggaacaccagtggcgaaggcggcctac |
| | | tgggcaccaactgacgctgaggctcgaaagtgtgggtagca |
| | | aacaggattagataccccggtagtc (SEQ ID No: 26) |
| 27 | Megamonas | Cctacggggggctgcagtggggaatcttccgcaatgggcga |
| | | aagcctgacggagcaacgccgcgtgaacgatgaaggtctta |
| | | ggatcgtaaagttctgttgttagggacgaagggtaagaatcat |
| | | aataaggtttttatttgacggtacctaacgaggaagccacggct |
| | | aactacgtgccagcagccgcggtaatacgtaggcggcaagc |
| | | gttgtccggaattattgggcgtaaagggagcgcaggcggga |
| | | aactaagcggatcttaaaagtgcggggctcaaccccgtgatg |
| | | gggtccgaactggttttcttgagtgcaggagaggaaagcgga |
| | | attcccagtgtagcggtgaaatgcgtagatattgggaagaac |
| | | accagtggcgaaggcggctttctggactgtaactgacgctga |
| | | ggctcgaaagctagggtagcgaacgggattagataccccag |
| | | tagtc (SEQ ID No: 27) |
| 28 | | Cctacggggggctgcagtggggaatcttgcgcaatgggggg |
| | | aaccctgacgcagcgacgccgcgtgcgggacgaaggccct |
| | | cgggtcgtaaaccgctttcagcagggaagaggccgaaaggt |
| | | gacggtacctgcagaagaagccccggctaaatacgtgccag |
| | | cagccgcggtaatacgtatggggcgagcgttatccggattcat |
| | | tgggcgtaaagcgcgcgtaggcggcctcgtaggccgggagt |
| | | caaatccgggggctcaacccccgcccgctcccggaacccc |
| | | gaggcttgagtctggcaggggagggtggaattcccagtgtag |
| | | cggtggaatgcgcagatattgggaagaacaccggtggcgaa |
| | | ggcggccctctgggccacgactgacgctgaggcgcgaaag |
| | | ctgggggagcgaacaggattagatacccgagtagtc (SEQ ID No: 28) |

TABLE 4
Descriptive statistics of control and IBS subjects studied

| | | Control | | IBS | |
|---|---|---|---|---|---|
| | | (n = 65) | | (n = 80) | |
| Age ranger years (mean) | | 19-65 | (45) | 17-66 | (39) |
| Sex (male/female) | | 15/49 | | 15/65 | |
| BMI Class, n 1%) | | | | | |
| | Normal | 25 | (38) | 31 | (39) |
| | Obese Class I | 11 | (17) | 14 | (18) |
| | Obese Class II | 3 | (5) | 5 | (6) |
| | Obese Class III | 1 | (2) | 3 | (4) |
| | Overweight | 21 | (22) | 22 | (22) |
| | Underweight | 3 | (3) | 3 | (4) |
| HADS: Anxiety, n 1%) | | | | | |
| | Normal (0-10) | 59 | (91) | 58 | (73) |
| | Abnormal | 6 | (9) | 22 | (28) |
| | (11-21) | | | | |
| HADS: Depression, n (%) | | | | | |
| | Normal (0-10) | 64 | (98) | 70 | (88) |
| | Abnormal | 1 | (2) | 10 | (13) |
| | (11-21) | | | | |
| Bristol Stool Score, n (%) | | | | | |
| | Normal | 54 | (83) | 18 | (23) |
| | Constipated | 8 | (12) | 22 | (28) |
| | Diarrhoea | 3 | (5) | 40 | (50) |
| IBS subtype, n 1%) | | | | | |
| | IBS-C | | 30 | (38) | |
| | IBS-D | N/A | 21 | (36) | |
| | IBS-M | | 29 | (36) | |
| SeHCAT assayed, n (%) | | 9 | (14) | 46 | (56) |
| Dietary group (FFQ), n 1%) | | | | | |
| | Omnivore | 63 | (97) | 74 | (93) |
| | Vegetarian | 1 | (2) | 2 | (3) |
| | Pescatarian | 1 | (2) | 1 | (1) |
| | Gluten-free | 0 | (0) | 4 | (5) |
| Drinks alcohol, n l%) | | | | | |
| | Current | 54 | (83) | 57 | (71) |
| | Previous | 0 | (0) | 1 | (1) |
| | Never | 10 | (15) | 22 | (28) |
| smoker, n (%) | | | | | |
| | Current | 10* | (15) | 14* | (18) |
| | Previous | 13 | (20) | 18 | (23) |
| | Never | 42 | (65) | 48 | (60) |

*1 subject in each group smoked e-cigarettes
N/A, not applicable

TABLE 5
Further 16S OTU Machine learning LASSO and Random Forest (RF) statistics

| LASSO | | | | RF | | | |
|---|---|---|---|---|---|---|---|
| lambda | AUC | Sens | Spec | mtry | AUC | Sens | Spec |
| 0.1 | 0.757 | 0.883 | 0.469 | 1 | 0.851 | 0.924 | 0.542 |

| Ten-fold cross-validation | | | Ten-fold cross-validation | | |
|---|---|---|---|---|---|
| Reference | | | Reference | | |
| Prediction | IBS | Healthy | Prediction | IBS | Healthy |
| IBS | 68.8 | 34.0 | IBS | 72.1 | 29.3 |
| Healthy | 9.2 | 30.0 | Healthy | 5.9 | 34.7 |
| Accuracy | (average) | 0.6958 | Accuracy | (average) | 0.7521 |

| RF Ranking | | | | | | |
|---|---|---|---|---|---|---|
| Rank # | Ranking | Phylum | Class | Order | Family | Genus |
| 1 | 100 | Firmicutes | Clostridia | Clostridiales | Lachnospiraceae | |
| 2 | 87.5 | Firmicutes | | | | |
| 3 | 82.1 | Firmicutes | Clostridia | Clostridiales | Ruminococcaceae | Butyricicoccus |
| 4 | 66.3 | Firmicutes | Clostridia | Clostridiales | Lachnospiraceae | |
| 5 | 62.4 | Firmicutes | Clostridia | Clostridiales | | |
| 6 | 57.2 | Firmicutes | Clostridia | Clostridiales | Ruminococcaceae | |
| 7 | 43.7 | Firmicutes | Clostridia | Clostridiales | Ruminococcaceae | |
| 8 | 30.8 | Firmicutes | | | | |
| 9 | 15.1 | Firmicutes | Clostridia | Clostridiales | Ruminococcaceae | |
| 10 | 0 | Firmicutes | Clostridia | Clostridiales | Lachnospiraceae | |

Analysis had 2 classes: Control and IBS and included 139 samples (IBS: n = 80 and Control: n = 59)
Metrics reported are the average values from 10 repeats of 10-fold Cross Validation.
Taxonomy classified using the RDP classfier, database version 2.10.1.

TABLE 6
Further 16S OTU Machine learning LASSO and Random Forest (RF)
statistics sequence information

| Rank # | Ranking | Phylum | Class | Order | Family |
|---|---|---|---|---|---|
| 1 | 100 | Firmicutes | Clostridia | Clostridiales | Lachnospiraceae |
| 2 | 87.5 | Firmicutes | | | |
| 3 | 82.1 | Firmicutes | Clostridia | Clostridiales | Ruminococcaceae |
| 4 | 66.3 | Firmicutes | Clostridia | Clostridiales | Lachnospiraceae |
| 5 | 62.4 | Firmicutes | Clostridia | Clostridiales | |
| 6 | 57.2 | Firmicutes | Clostridia | Clostridiales | Ruminococcaceae |
| 7 | 43.7 | Firmicutes | Clostridia | Clostridiales | Ruminococcaceae |
| 8 | 30.8 | Firmicutes | | | |
| 9 | 15.1 | Firmicutes | Clostridia | Clostridiales | Ruminococcaceae |
| 10 | 0 | Firmicutes | Clostridia | Clostridiales | Lachnospiraceae |

| Rank # | Genus | Sequence |
|---|---|---|
| 1 | | CCTACGGGGGGCAGCAGTGGGGAATATTGCACAATGGGGGAAACCCTG |
| | | ATGCAGCGACGCCGCGTGAGTGAAGAAGTATTTCGGTATGTAAAGCTCT |
| | | ATCAGCAGGGAAGAAAATGACGGTACCTGACTAAGAAGCCCCGGCTAAC |
| | | TACGTGCCAGCAGCCGCGGTAATACGTAGGGGGCAAGCGTTATCCGGAT |
| | | TTACTGGGTGTAAAGGGAGCGTAGGTGGTATGGCAAGTCAGAGGTGAA |
| | | AACCCAGGGCTTAACCTTGGGATTGCCTTTGAAACTGTCAGACTAGAGTG |
| | | CAGGAGGGGTAAGTGGAATTCCTAGTGTAGCGGTGAAATGCGTAGATAT |
| | | TAGGAGGAACACCAGTGGCGAAGGCGGCTTACTGGACTGTAACTGACAC |
| | | TGAGGCTCGAAAGCGTGGGGAGCAAACAGGATTAGATACCCGAGTAGT |
| | | C (SEQ ID No: 29) |
| 2 | | CCTACGGGGGGCTGCAGTGGGGAATATTGGGCAATGGAGGAAACTCTG |
| | | ACCCAGCAACGCCGCGTGGAGGAAGAAGGTTTTCGGATCGTAAACTCCT |
| | | GTCCTTGGAGACGAGTAGAAGACGGTATCCAAGGAGGAAGCCCCGGCT |
| | | AACTACGTGCCAGCAGCCGCGGTAATACGTAGGGGGCAAGCGTTGTCCG |
| | | GAATAATTGGGCGTAAAGGGCGCGTAGGCGGCTCGGTAAGTCTGGAGT |
| | | GAAAGTCCTGCTTTTAAGGTGGGAATTGCTTTGGATACTGTCGGGCTTGA |
| | | GTGCAGGAGAGGTTAGTGGAATTCCCAGTGTAGCGGTGAAATGCGTAG |
| | | AGATTGGGAGGAACACCAGTGGCGAAGGCGACTAACTGGACTGTAACT |
| | | GACGCTGAGGCGCGAAAGTGTGGGGAGCAAACAGGATTAGATACCCCA |
| | | GTAGTC (SEQ ID No: 30) |
| 3 | Butyricicoccus | CCTACGGGGGGCTGCAGTGGGGAATATTGCGCAATGGGGGAAACCCTG |
| | | ACGCAGCAACGCCGCGTGATTGAAGAAGGCCTTCGGGTTGTAAAGATCT |
| | | TTAATCAGGGACGAAACATGACGGTACCTGAAGAATAAGCTCCGGCTAA |
| | | CTACGTGCCAGCAGCCGCGGTAATACGTAGGGAGCAAGCGTTATCCGGA |
| | | TTTACTGGGTGTAAAGGGCGCGCAGGCGGGCCGGCAAGTTGGAAGTGA |
| | | AATCCGGGGGCTTAACCCCCGAACTGCTTTCAAAACTGCTGGTCTTGAGT |
| | | GATGGAGAGGCAGGCGGAATTCCGTGTGTAGCGGTGAAATGCGTAGAT |
| | | ATACGGAGGAACACCAGTGGCGAAGGCGGCCTGCTGGACATTAACTGAC |
| | | GCTGAGGCGCGAAAGCGTGGGGAGCAAACAGGATTAGATACCCCTGTA |
| | | GTC (SEQ ID No: 31) |
| 4 | | CCTACGGGTGGCTGCAGTGGGGAATATTGCACAATGGGGGAAACCCTG |
| | | ATGCAGCAACGCCGCGTGAGTGAAGAAGTATTTCGGTATGTAAAGCTCT |
| | | ATCAGCAGGAAAGAAAATGACGGTACCTGACTAAGAAGCCCCGGCTAAC |
| | | TACGTGCCAGCAGCCGCGGTAATACGTAGGGGGCAAGCGTTATCCGGAT |
| | | TTACTGGGTGTAAAGGGAGCGTAGACGGTGAGGCAAGTCTGAAGTGAA |
| | | ATGCCGGGGCTCAACCCCGGAACTGCTTTGGAAACTGTCGTACTAGAGT |
| | | GTCGGAGGGGTAAGCGGAATTCCTAGTGTAGCGGTGAAATGCGTAGAT |
| | | ATTAGGAGGAACACCAGTGGCGAAGGCGGCTTGCTGGACTGTAACTGAC |
| | | ACTGAGGCTCGAAAGCGTGGGGAGCAAACAGGATTAGATACCCTTGTAG |
| | | TC (SEQ ID No: 32) |
| 5 | | CCTACGGGGGGCAGCAGTCGGGAATATTGCGCAATGGAGGAAACTCTG |
| | | ACGCAGTGACGCCGCGTATAGGAAGAAGGTTTTCGGATTGTAAACTATT |
| | | GTCGTTAGGGAAGATACAAGACAGTACCTAAGGAGGAAGCTCCGGCTAA |
| | | CTACGTGCCAGCAGCCGCGGTAATACGTAGGGAGCAAGCGTTATCCGGA |
| | | TTTATTGGGTGTAAAGGGTGCGTAGACGGGACAACAAGTTAGTTGTGAA |
| | | ATCCCTCGGCTTAACTGAGGAACTGCAACTAAAACTATTGTTCTTGAGTG |
| | | TTGGAGAGGAAAGTGGAATTCCTAGTGTAGCGGTGAAATGCGTAGATAT |
| | | TAGGAGGAACACCGGTGGCGAAGGCGACTTTCTGGACAATAACTGACGT |
| | | TGAGGCACGAAAGTGTGGGGAGCAAACAGGATTAGATACCCCAGTAGT |
| | | C (SEQ ID No: 33) |
| 6 | | CCTACGGGGGGCTGCAGTGGGGAATATTGGGCAATGGGCGAAAGCCTG |
| | | ACCCAGCAACGCCGCGTGAAGGAAGAAGGTCTTCGGATTGTAAACTTCT |
| | | TTTATGAGGGACGAAGGAAGTGACGGTACCTCATGAATAAGCCACGGCT |
| | | AACTACGTGCCAGCAGCCGCGGTAATACGTAGGTGGCAAGCGTTGTCCG |
| | | GATTTACTGGGTGTAAAGGGCGCGTAGGCGGGATGGCAAGTCAGATGT |
| | | GAAATCCATGGGCTCAACCCATGAACTGCATTTGAAACTGTCGTTCTTGA |
| | | GTATCGGAGAGGCAAGCGGAATTCCTAGTGTAGCGGTGAAATGCGTAG |
| | | ATATTAGGAGGAACACCAGTGGCGAAGGCGGCTTGCTGGACGACAACT |
| | | GACGCTGAGGCGCGAAAGCGTGGGGAGCAAACAGGATTAGATACCCCT |
| | | GTAGTC (SEQ ID No: 34) |
| 7 | | CCTACGGGGGGCTGCAGTGGGGGATATTGCACAATGGGGGAAACCCTG |
| | | ATGCAGCAACGCCGCGTGAGGGAAGAAGGTTTTCGGATTGTAAACCTCT |
| | | GTCCTCAGGGAAGATAATGACGGTACCTGAGGAGGAAGCTCCGGCTAAC |
| | | TACGTGCCAGCAGCCGCGGTAATACGTAGGGAGCAAGCGTTGTCCGGAT |
| | | TTACTGGGTGTAAAGGGTGCGTAGGCGGGATATCAAGTCAGACGTGAA |
| | | ATCCATCGGCTTAACTGATGAACTGCGTTTGAAACTGGTATTCTTGAGTG |
| | | AGTCAGAGGCAGGCGGAATTCCCGGTGTAGCGGTGAAATGCGTAGAGA |
| | | TCGGGAGGAACACCAGTGGCGAAGGCGGCCTGCTGGGGCTTAACTGAC |
| | | GCTGAGGCACGAAAGCGTGGGGAGCAAACAGGATTAGATACCCGAGTA |
| | | GTC (SEQ ID No: 35) |
| 8 | | CCTACGGGGGGCTGCAGTGGGGAATATTGGGCAATGGAGGGAACTCTG |
| | | ACCCAGCAATGCCGCGTGAGTGAAGAAGGTTTTCGGATTGTAAAACTCTT |
| | | TAAGCAGGGACGAAGAAAGTGACGGTACCTGCAGAATAAGCATCGGCT |
| | | AACTACGTGCCAGCAGCCGCGGTAATACGTAGGATGCAAGCGTTATCCG |
| | | GAATGACTGGGCGTAAAGGGTGCGTAGGCGGTAAATCAAGTTGGCAGC |
| | | GTAATTCCGGGGCTTAACTCCGGAACTACTGCCAAAACTGGTGAACTAGA |
| | | GTGTGTCAGGGGTAAGTGGAATTCCTAGTGTAGCGGTGGAATGCGTAGA |
| | | TATTAGGAGGAACACCGGAGGCGAAAGCGACTTACTGGGGCACAACTG |
| | | ACGCTGAGGCACGAAAGCGTGGGGAGCAAACAGGATTAGATACCCCGG |
| | | TAGTC (SEQ ID No: 36) |
| 9 | | CCTACGGGAGGCAGCAGTGGGGGATATTGCACAATGGAGGAAACTCTG |
| | | ATGCAGCAACGCCGCGTGAGGGAAGAAGGATTTCGGTTTGTAAACCTCT |
| | | GTCTTCGGTGACGAAAATGACGGTAGCCGAGGAGGAAGCTCCGGCTAAC |
| | | TACGTGCCAGCAGCCGCGGTAATACGTAGGGAGCAAGCGTTGTCCGGAA |
| | | TTACTGGGTGTAAAGGGTGCGTAGGTGGGACTGCAAGTCAGGTGTGAA |
| | | AACGGTCGGCTCAACCGATCGCCTGCACTTGAAACTGTGGTTCTTGAGTG |
| | | AAGTAGAGGTAGGCGGAATTCCCGGTGTAGCGGTGAAATGCGTAGAGA |
| | | TCGGGAGGAACACCAGTGGCGAAGGCGGCCTACTGGGCTTTAACTGAC |
| | | GCTGAGGCACGAAAGCATGGGTAGCAAACAGGATTAGATACCCCGGTA |
| | | GTC (SEQ ID No: 37) |
| 10 | | CCTACGGGGGGCTGCAGTGGGGAATATTGCACAATGGGGGAAACCCTG |
| | | ATGCAGCGACGCCGCGTGAGCGAAGAAGTATTTCGGTATGTAAAGCTCT |
| | | ATCAGCAGGGAAGATAATGACGGTACCTGACTAAGAAGCCCCGGCTAAA |
| | | TACGTGCCAGCAGCCGCGGTAATACGTAGGGAGCAAGCGTTATCCGGAT |
| | | TTATTGGGTGTAAAGGGTGCGTAGACGGGACAACAAGTTAGTTGTGAAA |
| | | TCCCTCGGCTTAACTGAGGAACTGCAACTAAAACTATTGTTCTTGAGTGTT |
| | | GGAGAGGAAAGTGGAATTCCTAGTGTAGCGGTGAAATGCGTAGATATTA |
| | | GGAGGAACACCGGTGGCGAAGGCGGCCTACTGGGCACCAACTGACGCT |
| | | GAGGCTCGAAAGTGTGGGTAGCAAACAGGATTAGATACCCTAGTAGTC |
| | | (SEQ ID No: 38) |

TABLE 7
Fecal Metabolomics Machine learning using the alternative machine learning pipeline is predictive
of BAM status in IBS

| | LASSO | Random Forest | Model |
|---|---|---|---|
| | Optimisation | Optimisation | Performance |
| AUC | 0.878 (0.023) | 0.976 (0.013) | 0.8521 |
| Sensitivity | 0.827 (0.055) | 0.911 (0.038) | 0.684 |
| Specificity | 0.77 (0.051) | 0.9 (0.044) | 0.762 |

10-fold Cross Validation

| | Predicted BAM | Predicted Normal | |
|---|---|---|---|
| BAM | 13 | 6 | |
| Normal | 5 | 16 | |
| | | LASSO | Random Forest |
| Rank # | Metabolite ID | coefficients | feature importance |
| 1 | 1,3-di-(5Z,8Z,11Z,14Z,17Z- | 0.9282 | 85.74 |
| | eicosapentaenoyl)-2-hydroxy- | | |
| | glycerol (d5) | | |
| 2 | Dimethyl benzyl carbinyl butyrate | 0.7124 | 68.62 |
| | 1-18:0-2-18:2- | −0.4293 | 62.96 |
| 3 | monogalactosyldiacylglycerol | | |
| 4 | PG(P-16:0/14:0) | 0.2362 | 62.26 |
| 5 | Glu-Glu-Gly-Tyr | −1.6603 | 60.75 |
| 6 | PC(22:2(13Z,16Z)/15:0) | −0.6728 | 56.95 |
| 7 | PG(34:0) | 0.1836 | 55.17 |
| 8 | PE(18:3(6Z,9Z,12Z)/P-18:0) | 0.227 | 48.27 |
| 9 | MG(22:2(13Z,16Z)/0:0/0:0) | −0.0225 | 18.77 |
| 10 | Arg-Ile-Gln-Ile | −0.2958 | 15.62 |
| 11 | PE(22:5(7Z,10Z,13Z,16Z,19Z)/24:0) | −0.2327 | 12.24 |
| 12 | PC(18:1(9Z)/15:0) | −0.2305 | 11.21 |
| 13 | Thiophanate-methyl | 0.0516 | 8.22 |
| 14 | Asn-Ser-His-His | −0.0173 | 8.15 |
| 15 | 1,2,3-Tris(1-ethoxyethoxy)propane | −0.0321 | 7.12 |
| 16 | PS(39:6) | 0.017 | 6.28 |
| 17 | 2-Hydroxylauroylcarnitine | −0.005 | 4.72 |
| 18 | Hypoxanthine | −0.0125 | 4.66 |
| 19 | Adenosine | −0.019 | 4.34 |
| 20 | PC(40:6) | −0.0486 | 3.93 |
| 21 | Asp-Phe-Phe-Val | 0.0438 | 3.45 |
| 22 | 3-Dehydroxycarnitine | −0.0165 | 2.83 |
| 23 | Inosine | −0.0035 | 2.17 |
| 24 | PG(O-34:3) | 0.0071 | 1.74 |
| 25 | 11-Deoxocucurbitacin I | −0.0112 | 1.64 |
| 26 | Methyl caprate | −0.0001 | 1.31 |
| 27 | Linoleoyl ethanolamide | −0.0025 | 1.06 |
| 28 | His-Met-Phe-Phe | −0.0079 | 1 |
| 29 | 1-Decanol | 0.0054 | 0.96 |
| 30 | Gravelliferone | −0.0129 | 0.65 |
| 31 | Uridine | −0.0098 | 0.64 |
| 32 | Arachidyl carnitine | −0.0036 | 0.62 |
| 33 | Guanosine | −0.0091 | 0.59 |
| 34 | Methyl nonylate | −0.0006 | 0.53 |
| 35 | 3-Epidemissidine | 0.0011 | 0.49 |
| 36 | Momordol | 0.0012 | 0.41 |
| 37 | N-[2-(1H-Indol-3- | −0.0334 | 0.41 |
| | yl)ethyl]docosanamide | | |
| 38 | Methyl caproate | −0.0044 | 0.34 |
| 39 | Ascorbic acid | −0.0148 | 0.32 |
| 40 | N-Acetyl-leu-leu-tyr | −0.0006 | 0.06 |
| 41 | 4-Hydroxybutyric acid | −0.0009 | 0.03 |
| | [ST dimethyl(4:0/3:0)] (5Z,7E,17Z)- | −0.0066 | 0 |
| | (1S,3R)-26,27-dimethyl-9,10-seco- | | |
| | 5,7,10(19),17(20)-cholestatetraen- | | |
| 42 | 22-yne-1,3,25-triol | −0.0009 | 0 |
| | N-Methylindolo[3,2-b]-5alpha- | | |
| 43 | cholest-2-ene | −0.0002 | 0 |
| | gamma-Glutamyl-S- | | |
| 44 | methylcysteinyl-beta-alanine | | |

LASSO and Random Forest (RF) statistics of metabolites predictive of BAM status
Analysis had 2 classes: BAM and Normal included 40 IBS samples (BAM: n = 19 and Normal: n = 21)
Metrics reported are the mean and the standard deviation of values from Cross Validation.

### REFERENCES

1. Slattery S A, Niaz O, Aziz Q, et al. Systematic review with meta-analysis: the prevalence of bile acid malabsorption in the irritable bowel syndrome with diarrhoea. Aliment. Pharmacol. Ther. 2015; 42:3-11.
2. Wildt S, Norby Rasmussen S, Lysgard Madsen J, et al. Bile acid malabsorption in patients with chronic diarrhoea: clinical value of SeHCAT test. Scand. J. Gastroenterol. 2003; 38:826-30.
3. Damsgaard B, Dalby H R, Krogh K, et al. Long-term effect of medical treatment of diarrhoea in 377 patients with SeHCAT scan diagnosed bile acid malabsorption from 2003 to 2016; a retrospective study. Aliment. Pharmacol. Ther. 2018; 47:951-957.
4. Camilleri M. Bile Acid diarrhea: prevalence, pathogenesis, and therapy. Gut Liver. 2015; 9(3):332-9.
5. Wedlake L, A'Hern R, Russell D, Thomas K, Walters J R, Andreyev H J. 2009. Systematic review: the prevalence of idiopathic bile acid malabsorption as diagnosed by SeHCAT scanning in patients with diarrhoea-predominant irritable bowel syndrome. Aliment Pharmacol Ther 30:707-717.
6. Thaysen E H, Orholm M, Arnfred T, Carl J, Rodbro P. 1982. Assessment of ileal function by abdominal counting of the retention of a gamma emitting bile acid analogue. Gut 23:862-865.
7. Enck P, Aziz Q, Barbara G, et al. Irritable bowel syndrome. Nat. Rev. Dis. Primers 2016; 2:16014.
8. Soares R L. Irritable bowel syndrome: a clinical review. World J. Gastroenterol. 2014; 20:12144-60.
9. Van Oudenhove L, Aziz Q. The role of psychosocial factors and psychiatric disorders in functional dyspepsia. Nat. Rev. Gastroenterol. Hepatol. 2013; 10:158-67.
10. Koloski N A, Jones M, Kalantar J, et al. The brain-gut pathway in functional gastrointestinal disorders is bidirectional: a 12-year prospective population-based study. Gut 2012; 61:1284-90.
11. Schwille-Kiuntke J, Mazurak N, Enck P. Systematic review with meta-analysis: post-infectious irritable bowel syndrome after travellers' diarrhoea. Aliment. Pharmacol. Ther. 2015; 41:1029-37.
12. Quigley E M M. The Gut-Brain Axis and the Microbiome: Clues to Pathophysiology and Opportunities for Novel Management Strategies in Irritable Bowel Syndrome (IBS). J. Clin. Med. 2018; 7.
13. Drossman D A, Morris C B, Schneck S, et al. International survey of patients with IBS: symptom features and their severity, health status, treatments, and risk taking to achieve clinical benefit. J. Clin. Gastroenterol. 2009; 43:541-50.
14. Lacy B E, Everhart K K, Weiser K T, et al. IBS patients' willingness to take risks with medications. Am. J. Gastroenterol. 2012; 107:804-9.
15. Carroll I M, Ringel-Kulka T, Keku T O, et al. Molecular analysis of the luminal- and mucosalassociated intestinal microbiota in diarrhea-predominant irritable bowel syndrome. Am. J. Physiol. Gastrointest. Liver Physiol. 2011; 301:G799-807.
16. Rajilic-Stojanovic M, Biagi E, Heilig H G, et al. Global and Deep Molecular Analysis of Microbiota Signatures in Fecal Samples From Patients With Irritable Bowel Syndrome. Gastroenterology 2011; 141:1792-1801.
17. Jeffery I B, O'Toole P W, Ohman L, et al. An irritable bowel syndrome subtype defined by species-specific alterations in faecal microbiota. Gut 2012; 61:997-1006.
18. Tap J, Derrien M, Tornblom H, et al. Identification of an Intestinal Microbiota Signature Associated With Severity of Irritable Bowel Syndrome. Gastroenterology 2017; 152:111-123 e8.
19. Collins S M. A role for the gut microbiota in IBS. Nat. Rev. Gastroenterol. Hepatol. 2014; 11:497-505.
20. Ohman L, Simren M. Intestinal microbiota and its role in irritable bowel syndrome (IBS). Curr. Gastroenterol. Rep. 2013; 15:323.
21. Longstreth G F, Thompson W G, Chey W D, et al. Functional bowel disorders. Gastroenterology 2006; 130:1480-91.
22. Zigmond, A. S. and R. P. Snaith, The hospital anxiety and depression scale. Acta Psychiatr. Scand., 1983. 67(6): p. 361-70.
23. Power, S. E., et al., Food and nutrient intake of Irish community-dwelling elderly subjects: who is at nutritional risk? J. Nutr. Health Aging., 2014. 18(6): p. 561-72.
24. Brown J R, Flemer B, Joyce S A, et al. Changes in microbiota composition, bile and fatty acid metabolism, in successful faecal microbiota transplantation for Clostridioides difficile infection. BMC Gastroenterol. 2018; 18:131.
25. Magoc T, Salzberg S L. FLASH: fast length adjustment of short reads to improve genome assemblies. Bioinformatics 2011; 27:2957-63.
26. Edgar R C. Search and clustering orders of magnitude faster than BLAST. Bioinformatics 2010; 26:2460-1.
27. Edgar R C. UPARSE: highly accurate OTU sequences from microbial amplicon reads. Nat. Methods 2013; 10:996-8.
28. Edgar R C, Haas B J, Clemente J C, et al. UCHIME improves sensitivity and speed of chimera detection. Bioinformatics 2011; 27:2194-200.
29. Consortium H M P. The Human Microbiome Project Consortium. Structure, function and diversity of the healthy human microbiome. Nature 2012; 486:207-14.
30. Franzosa E A, McIver L J, Rahnavard G, et al. Species-level functional profiling of metagenomes and metatranscriptomes. Nat. Methods 2018; 15:962-968.
31. Watson L, Lalji A, Bodla S, et al. Management of bile acid malabsorption using low-fat dietary interventions: a useful strategy applicable to some patients with diarrhoea-predominant irritable bowel syndrome? Clin. Med. (Lond.) 2015; 15:536-40.
32. Flemer B, Warren R D, Barrett M P, et al. The oral microbiota in colorectal cancer is distinctive and predictive. Gut 2018; 67:1454-1463.
33. Kurien M, Thurgar E, Davies A, et al. Challenging current views on bile acid diarrhoea and malabsorption. Frontline Gastroenterol. 2018; 9:92-97.
34. Summers J A, Peacock J, Coker B, et al. Multicentre prospective survey of SeHCAT provision and practice in the U K. BMJ Open Gastroenterol. 2016; 3:e000091.
35. Zhou C, Jia H M, Liu Y T, et al. Metabolism of glycerophospholipid, bile acid and retinol is correlated with the early outcomes of autoimmune hepatitis. Mol. Biosyst. 2016; 12:1574-85.
36. Huang H J, Zhang A Y, Cao H C, et al. Metabolomic analyses of faeces reveals malabsorption in cirrhotic patients. Dig. Liver Dis. 2013; 45:677-82.
37. Falony G, Joossens M, Vieira-Silva S, et al. Population-level analysis of gut microbiome variation. Science 2016; 352:560-4.
38. Ford A C, Harris L A, Lacy B E, et al. Systematic review with meta-analysis: the efficacy of prebiotics, probiotics, synbiotics and antibiotics in irritable bowel syndrome. Aliment. Pharmacol. Ther. 2018; 48:1044-1060.
39. Blaxter, M.; Mann, J.; Chapman, T.; Thomas, F.; Whitton, C.; Floyd, R.; Abebe, E. (October 2005). “Defining operational taxonomic units using DNA barcode data”. Philos Trans R Soc Lond B Biol Sci. 360 (1462): 1935-43.

1.-15. (canceled)

16. A method comprising:
detecting in a biological sample from a subject the level of (i) a bacterial strain of a taxa associated with bile acid malabsorption (BAM) or (ii) a metabolite associated with BAM, a precursor thereof, or a breakdown product thereof, and comparing the detected level of (i) or (ii) with the corresponding level of (i) or (ii) in a biological sample from a subject that does not have BAM,
wherein the subject is determined to have BAM when there is an increase in the detected level of (i) or (ii) compared to the corresponding level of (i) or (ii) in the biological sample from the subject that does not have BAM.

17. The method of claim 16, wherein the detecting of the bacterial strain further comprises 16S amplicon sequencing or shotgun sequencing.

18. The method of claim 16, wherein the detecting of the metabolite further comprises performing gas chromatography and liquid chromatography mass spectrometry (GC/LC MS).

19. The method of claim 16, wherein the bacterial strain is of the family selected from the group consisting of Lachnospiraceae, Bacteroidaceae, Ruminococcaceae, Bifidobacteriaceae, Prevotellaceae, Veillonellaceae, and Coriobacteriaceae.

20. The method of claim 16, wherein the bacterial strain is of the genus selected from the group consisting of Blautia, Bacteroides, Faecalibacterium, Oscillibacter, Ruminococcus, Bifidobacterium, Coprococcus, Paraprevotella, Gemmiger, Dialister, Megamonas, and Butyricicoccus.

21. The method of claim 16, wherein the bacterial strain belongs to an operational taxonomic unit (OTU) selected from Table 2 or Table 5.

22. The method of claim 16, wherein the bacterial strain has a 16S rRNA gene sequence having at least 97% sequence identity to any one of SEQ ID NOs: 1-38.

23. The method of claim 16, further comprising detecting two or more bacterial strains of two or more bacterial taxa associated with BAM.

24. The method of claim 16, wherein the metabolite is selected from the group consisting of PG(P-16:0/14:0), 2-Ethylsuberic acid, Glu-Glu-Gly-Tyr, 1,2,3-Tris(l-ethoxyethoxy)propane, PG(O-30:1), Ursodeoxycholic acid, MG(22:2(13Z,16Z)/0:0/0:0), L-Lysine, O-Phosphoethanolamine, PE(22:5(7Z,10Z,13Z,16Z,19Z)/24:0), and Heptadecanoic acid.

25. The method of claim 16, wherein the metabolite is selected from the group consisting of 1,3-di-(5Z,8Z,11Z,14Z,17Z-eicosapentaenoyl)-2-hydroxy-glycerol (d5), Dimethyl benzyl carbinyl butyrate, 1-18:0-2-18:2-monogalactosyldiacylglycerol, PG(P-16:0/14:0), Glu-Glu-Gly-Tyr, PC(22:2(13Z,16Z)/15:0), PG(34:0), PE(18:3(6Z,9Z,12Z)/P-18:0), MG(22:2(13Z,16Z)/0:0/0:0), Arg-Ile-Gln-Ile, PE(22:5(7Z,10Z,13Z,16Z,19Z)/24:0), PC(18:1(9Z)/15:0), Thiophanate-methyl, Asn-Ser-His-His, 1,2,3-Tris(l-ethoxyethoxy)propane, PS(39:6), 2-Hydroxylauroylcarnitine, Hypoxanthine, Adenosine, PC(40:6), Asp-Phe-Phe-Val, 3-Dehydroxycarnitine, Inosine, PG(0-34:3), 11-Deoxocucurbitacin I, Methyl caprate, Linoleoyl ethanolamide, His-Met-Phe-Phe, 1-Decanol, Gravelliferone, Uridine, Arachidyl carnitine, Guanosine, Methyl nonylate, 3-Epidemissidine, Momordol, N-[2-(lH-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E, 17Z)-(1S,3R)-26,27-dimethyl-9, 10-seco-5,7, 10(19), 17(20)-cholestatetraen-22-yne-1,3,25-triol, N-Methylindolo[3,2-b]-5alpha-cholest-2-ene, and gamma-Glutamyl-S-methylcysteinyl-beta-alanine.

26. The method of claim 16, wherein the metabolite is selected from the group consisting of PG(P-16:0/14:0), 2-Ethylsuberic acid, Glu-Glu-Gly-Tyr, 1,2,3-Tris(l-ethoxyethoxy)propane, PG(O-30:1), Ursodeoxycholic acid, MG(22:2(13Z,16Z)/0:0/0:0), L-Lysine, O-Phosphoethanolamine, PE(22:5(7Z,10Z,13Z,16Z,19Z)/24:0), Heptadecanoic acid, 1,3-di-(5Z,8Z,HZ,14Z,17Z-eicosapentaenoyl)-2-hydroxy-glycerol (d5), Dimethyl benzyl carbinyl butyrate, 1-18:0-2-18:2-monogalactosyldiacylglycerol, PC(22:2(13Z,16Z)/15:0), PG(34:0), PE(18:3(6Z,9Z,12Z)/P-18:0), Arg-Ile-Gln-Ile, PC(18:1(9Z)/15:0), Thiophanate-methyl, Asn-Ser-His-His, PS(39:6), 2-Hydroxylauroylcarnitine, Hypoxanthine, Adenosine, PC(40:6), Asp-Phe-Phe-Val, 3-Dehydroxycarnitine, Inosine, PG(0-34:3), 11-Deoxocucurbitacin I, Methyl caprate, Linoleoyl ethanolamide, His-Met-Phe-Phe, 1-Decanol, Grave lliferone, Uridine, Arachidyl carnitine, Guanosine, Methyl nonylate, 3-Epidemissidine, Momordol, N-[2-(lH-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E,17Z)-(lS,3R)-26,27-dimethyl-9,10-seco-5,7,10(19),17(20)-cholestatetraen-22-yne-1,3,25-triol, N-Methylindolo[3,2-b]-5alpha-cholest-2-ene, and gamma-Glutamyl-S-methylcysteinyl-beta-alanine.

27. The method of claim 16, wherein the subject has been previously diagnosed with irritable bowel syndrome (IBS), anorexia nervosa, or inflammatory bowel disease (IBD).

28. The method of claim 16, wherein the biological sample comprises a fecal sample, a urine sample, or an oral sample.

29. The method of claim 16, wherein the subject is a human.

30. The method of claim 16, wherein the method further comprises treating the subject determined to have BAM.

31. The method of claim 30, wherein the treatment comprises administering to the subject a bile acid sequestrant, loperamide, a laxative, an antidepressant, a fecal transplant, an antibiotic, a probiotic, or a live biotherapeutic.

32. A method of treating bile acid malabsorption (BAM) in a subject in need thereof comprising administering to the subject a treatment for BAM selected from a bile acid sequestrant, loperamide, a laxative, an antidepressant, a fecal transplant, an antibiotic, a probiotic, or a live biotherapeutic after detecting in a biological sample from the subject an elevated level of (i) a bacterial strain of a taxa associated with BAM or (ii) a metabolite associated with BAM, a precursor thereof, or a breakdown product thereof, as compared to the corresponding level of (i) or (ii) in a biological sample from a subject that does not have BAM.

33. The method of claim 32, wherein the bacterial strain is of the family selected from the group consisting of Lachnospiraceae, Bacteroidaceae, Ruminococcaceae, Bifidobacteriaceae, Prevotellaceae, Veillonellaceae, and Coriobacteriaceae or is from the genus selected from the group consisting of Blautia, Bacteroides, Faecalibacterium, Oscillibacter, Ruminococcus, Bifidobacterium, Coprococcus, Paraprevotella, Gemmiger, Dialister, Megamonas, and Butyricicoccus.

34. The method of claim 32, wherein the metabolite is selected from the group consisting of 1,3-di-(5Z,8Z,11Z,14Z,17Z-eicosapentaenoyl)-2-hydroxy-glycerol (d5), Dimethyl benzyl carbinyl butyrate, 1-18:0-2-18:2-monogalactosyldiacylglycerol, PG(P-16:0/14:0), Glu-Glu-Gly-Tyr, PC(22:2(13Z,16Z)/15:0), PG(34:0), PE(18:3(6Z,9Z,12Z)/P-18:0), MG(22:2(13Z,16Z)/0:0/0:0), Arg-Ile-Gln-Ile, PE(22:5(7Z,10Z,13Z,16Z,19Z)/24:0), PC(18:1(9Z)/15:0), Thiophanate-methyl, Asn-Ser-His-His, 1,2,3-Tris(l-ethoxyethoxy)propane, PS(39:6), 2-Hydroxylauroylcarnitine, Hypoxanthine, Adenosine, PC(40:6), Asp-Phe-Phe-Val, 3-Dehydroxycarnitine, Inosine, PG(0-34:3), 11-Deoxocucurbitacin I, Methyl caprate, Linoleoyl ethanolamide, His-Met-Phe-Phe, 1-Decanol, Gravelliferone, Uridine, Arachidyl carnitine, Guanosine, Methyl nonylate, 3-Epidemissidine, Momordol, N-[2-(lH-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E, 17Z)-(1S,3R)-26,27-dimethyl-9, 10-seco-5,7, 10(19), 17(20)-cholestatetraen-22-yne-1,3,25-triol, N-Methylindolo[3,2-b]-5alpha-cholest-2-ene, and gamma-Glutamyl-S-methylcysteinyl-beta-alanine, PG(P-16:0/14:0), 2-Ethylsuberic acid, Glu-Glu-Gly-Tyr, 1,2,3-Tris(l-ethoxyethoxy)propane, PG(O-30:1), Ursodeoxycholic acid, MG(22:2(13Z,16Z)/0:0/0:0), L-Lysine, O-Phosphoethanolamine, PE(22:5(7Z,10Z,13Z,16Z,19Z)/24:0), Heptadecanoic acid, 1,3-di-(5Z,8Z,HZ,14Z,17Z-eicosapentaenoyl)-2-hydroxy-glycerol (d5), Dimethyl benzyl carbinyl butyrate, 1-18:0-2-18:2-monogalactosyldiacylglycerol, PC(22:2(13Z,16Z)/15:0), PG(34:0), PE(18:3(6Z,9Z,12Z)/P-18:0), Arg-Ile-Gln-Ile, PC(18:1(9Z)/15:0), Thiophanate-methyl, Asn-Ser-His-His, PS(39:6), 2-Hydroxylauroylcarnitine, Hypoxanthine, Adenosine, PC(40:6), Asp-Phe-Phe-Val, 3-Dehydroxycarnitine, Inosine, PG(0-34:3), 11-Deoxocucurbitacin I, Methyl caprate, Linoleoyl ethanolamide, His-Met-Phe-Phe, 1-Decanol, Grave lliferone, Uridine, Arachidyl carnitine, Guanosine, Methyl nonylate, 3-Epidemissidine, Momordol, N-[2-(lH-Indol-3-yl)ethyl]docosanamide, Methyl caproate, Ascorbic acid, N-Acetyl-leu-leu-tyr, 4-Hydroxybutyric acid, [ST dimethyl(4:0/3:0)] (5Z,7E,17Z)-(lS,3R)-26,27-dimethyl-9,10-seco-5,7,10(19),17(20)-cholestatetraen-22-yne-1,3,25-triol, N-Methylindolo[3,2-b]-5alpha-cholest-2-ene, and gamma-Glutamyl-S-methylcysteinyl-beta-alanine.

35. A kit comprising reagents for detecting:
(a) a bacterial strain of a taxa associated with bile acid malabsorption (BAM); and
(b) a metabolite associated with BAM.
